(19) United States
(12) Patent Application Publication
(10) Pub. No.: US 2009/0312675 A1
(43) Pub. Date: Dec. 17, 2009

(54) SUB-ATMOSPHERIC PRESSURE CHAMBER FOR MECHANICAL ASSISTANCE OF BLOOD FLOW

(75) Inventors: Christian E. Sampson, Chestnut Hill, MA (US); Loutfallah Georges Chedid, West Newton, MA (US); Adam Ysasi, Seattle, WA (US); Dennis Zomar, Marlborough, MA (US); Christopher Scully, Groton, CT (US); Lorenzo Serra, South Salem, NY (US)

Correspondence Address: FOLEY HOAG, LLP;PATENT GROUP, WORLD TRADE CENTER WEST, 155 SEAPORT BLVD, BOSTON, MA, 02110, US

(73) Assignee: The Brigham and Women's Hospital, Inc., Boston, MA (US)

(21) Appl. No.: 12/374,442

(22) PCT Filed: Jul. 19, 2007
(86) PCT No.: PCT/US07/73941

§ 371 Date: Jan. 20, 2009

Related U.S. Application Data
(60) Provisional application No. 60/832,240, filed on Jul. 19, 2006.

(51) Int. Cl. A61H 7/00; A61M 1/00

(52) U.S. Cl. 601/10; 601/11

(57) ABSTRACT

A sub-atmospheric pressure treatment device may include a chamber, a vacuum source, a pulse sensor, and a controller.

### CROSS-REFERENCE TO RELATED APPLICATION

This application claims the benefit of U.S. provisional application Ser. No. 60/832,240, filed Jul. 19, 2006, which is hereby incorporated herein by reference.

### SUMMARY

An electromechanical device was desired to remedy the effects of poor circulation in an extremity. Problems in circulation in an extremity can be caused by a loss of blood or diseases such as sepsis where the central body organs horde the blood. This can lead to damage or to loss of an extremity. It is believed that by reducing the atmospheric pressure around an extremity in a cyclical pattern, blood flow will increase and the extremity can be saved before life-changing surgery must take place. A device was developed that uses the input signals from a Radical™ pulse oximeter to control the pressure inside of a chamber enclosing the hand and a portion of the forearm. The vacuum gage pressure in the chamber will be dropped at least 25 mmHg, such as 75 mmHg or 100 mmHg, when the vacuum is desired, and then it will return to atmospheric pressure for a short period of time. There are three different modes that may be used to cycle the pressure in the chamber. The pulse synchronization mode uses the Pulse Rate output from a Radical pulse oximeter to determine the time that the pressure should be dropped in the chamber. For this program, it is desired that the pressure be reduced as blood is flowing into the hand, and that pressure return to atmosphere as not to hinder venous return. The Signal IQ® output from the Radical pulse oximeter is used to trigger this cycle. It is also used to trigger the alternating pulse mode which reduces the pressure for a full pulse, and then the chamber is returned to atmosphere for a set number of pulses. The third program developed was a time based program. Initial investigations with the completed device prove promising. An increase in the area underneath the Pleth waveform is seen when the pulse synchronization mode is used. This is believed to be caused by an increase in blood flow in the hand from the cyclical drop in pressure around the hand.

### BRIEF DESCRIPTION OF THE DRAWINGS

FIG. 1 schematically depicts an exemplary embodiment of a subatmospheric pressure treatment device.

FIG. 2 depicts an embodiment of a chamber and flange.

FIG. 3 depicts pleth waveform vs. Signal IQ output.

FIG. 4 depicts pulse rate serial output data.

FIGS. 5-8 depict program module flow charts.

FIG. 9 depicts a diagram of microcontroller inputs and outputs.

FIGS. 10-16 depict schematics for various portions of a controller.

FIG. 17 depicts a microcontroller schematic.

FIG. 18 depicts an embodiment of a case.

FIGS. 19-30 depict details of an embodiment of a chamber and flange.

FIGS. 31-41 depict details of an embodiment of a case.

FIGS. 42-44 depict various features of data acquisition systems.

### DETAILED DESCRIPTION

### 1. Introduction

The manifestation of vascular disease in the extremity is a spectrum from symptoms of claudication, to ulcerations, and finally, limb-threatening ischemia. Arterial disease causes upper extremity ischemia in approximately 5% of cases with the remaining 95% being lower extremity ischemia. Atherosclerosis, emboli, thoracic outlet syndrome, subclavian steal, Raynaud's disease, aneurysms, and Buerger's disease are some of the types of arterial disease affecting perfusion of the extremity. Other co-morbidities such as diabetes worsen the effects of extremity ischemia. One condition known for its disastrous effect on the upper and lower extremity is Purpura Fulminans (PF), a condition usually associated with sepsis. Features include a rapidly progressive tissue necrosis, small vessel thrombosis and disseminated intravascular coagulation. If treatment with antibiotics and other supportive measures is begun early, survival is likely, but it remains a disabling condition often requiring major amputations of the upper and lower extremities. In such cases, medical treatment to support blood pressure and perfusion of the central organs further compromises perfusion of the extremities, resulting in more tissue loss. In addition to those conditions causing acute ischemia, there are many cases of chronic ischemia affecting the extremity and which cannot by treated adequately by medical or surgical means.

Devices are disclosed herein which can be used to support tissue perfusion in the extremities during the acute phase of any disease which compromises tissue perfusion. Such a device works by altering the balance of forces determining blood flow. Blood flow in capillaries is described by Starling's equilibrium, a formula which takes into account blood capillary pressure, colloidosmotic pressure in both the capillary, and interstitial tissue, and tissue pressure. Tissue pressure is determined by atmospheric pressure and it is this component of Starling's equilibrium affected by our device. Reducing the atmospheric pressure the extremity is exposed to favors perfusion by reducing the force blood capillary pressure needs to overcome.

When a body undergoes a trauma or ailment, localized or body wide, blood is reserved for vital organs, causing a loss of blood flow to the body's extremities. There the need exists to maintain and improve peripheral blood circulation in order to prevent damage or loss of the extremities. Loss of blood or diseases such as sepsis or septicemia can cause poor blood circulation to the extremities, risking damage or loss of the extremities of the body.

If blood flow can be increased to the extremities of the body during the critical stages of trauma or ailment, the risk of loss of the extremities decreases greatly. The hypothesis is that by exposing the extremity to a sub-atmospheric pressure the poor blood flow can be increased.

In order to achieve a sub-atmospheric pressure around an extremity a sealed rigid chamber was constructed with a near instantaneous supply of vacuum. This is to be supplied in a cyclical process in order to not restrict venous return. Creating an absolute pressure of 685-735 mmHg inside the chamber will provide a low enough pressure for peripheral circulation to be maintained.

In order to test the effects of sub-atmospheric pressure on peripheral circulation a prototype was designed and constructed.

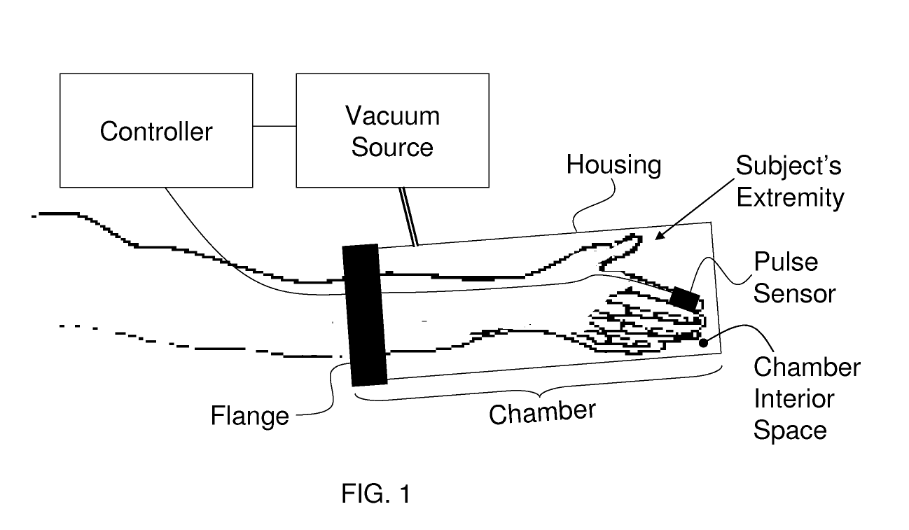

FIG. 1 schematically depicts an embodiment of a sub-atmospheric pressure treatment device, including a chamber having a flange, housing, and interior space, a pulse sensor attached to a subject's extremity, a vacuum source in communication with the housing interior space, and a controller receiving signals from the pulse sensor and controlling the vacuum source.

### 2. Problem Definition

By reducing pressure around an extremity in the range of −25 mmHg to −75 mmHg it is thought that blood flow will improve. This would allow the extremity to survive through the most desperate medical times and after recovery. Pressure must be reduced cyclically, releasing back to atmospheric pressure or else too much blood will gather in the surrounded area. This cyclical process is predicted to have the most beneficial results when linked with a person's pulse. Sub-atmospheric pressure conditions would be created when blood is being pumped into the extremity by the heart, and then atmospheric pressure would surround the extremity for the venous return.

To achieve a sub-atmospheric pressure around an extremity a sealed rigid transparent chamber containing a minimal amount of volume was constructed. A near instantaneous supply of vacuum needs to be applied to the chamber in a cyclical process synchronized to the patient's heartbeat in order to not restrict venous return. The on cycle absolute pressure must be lowered to 685-735 mmHg and then the off cycle must return the chamber to atmospheric pressure. The patient's blood flow and pulse may be monitored while the extremity is sealed in the chamber. A microcontroller will be used to synchronize the patient's heart beat with the on-off cycle of the vacuum source and monitor the pressure within the chamber.

### 3. Design

A. Chamber Design

During initial conception of the sub-atmospheric pressure chamber design several functional requirements were set. The chamber would have to be cylindrical with a minimal diameter to reduce the volume of air within the chamber. It would have to be transparent so the patient's hand and arm could be monitored visually. It would also have to be rigid so the chamber would not collapse under vacuum, and air-tight for maximum efficiency. The flange that would be used to seal the chamber to the patient's arm would have to be flexible and create an air-tight seal. The flange would also have to apply enough pressure to the patient's arm to create an air-tight seal and at the same time not impede venous return.

The chamber may be constructed in a variety of ways. In a “push-through” arrangement, the patient's hand and arm is inserted directly through the flange of the chamber, and the flange seals to the patient's forearm just below the elbow. In an “open case” arrangement, the cylindrical chamber is formed from two halves that are hinged on one side. The sides that were not hinged would have latches which would induce pressure to a rubber gasket creating an air-tight seal. The open-case arrangement can also include a flange as described above. The “push-through” arrangement is discussed further.

Once the initial design was finalized, specific dimensions of the chamber needed to be set. The size of the chamber was based on the hand and forearm dimensions averaged from several 20-25 year old males with a weight of 140-200 lbs and a height of 5′-8″ to 6′-4″. The chamber would need a wall thickness of 3/16″ for maximum durability. The diameter of the chamber was selected to be 5½″ so the patient's hand would be in a relaxed position increasing the comfort level. The 18″ length of the chamber was established by taking into account the room necessary for the pulse oximeter probe to be connected to the patient's finger.

Following determination of the functional requirements and the selected dimensions of the chamber, materials were selected for the chamber. Several choices of case material were available, but limited by the size, availability, and cost. Three materials were selected to be considered for the chamber: acrylic plastic, lexan, and clear PVC. Several strengths and weaknesses were established for each material after they were researched for their mechanical characteristics, prices, and availability. Lexan tubing was found to be very rare since most industrial applications that require cylindrical transparent durable material use acrylic plastic. Clear PVC was found to be readily available and low-cost at sixteen to twenty-two dollars a foot depending on the supplier. After contacting several suppliers, it was discovered that clear PVC was only available in 4″ and 6″ diameters and 10′ lengths. With these facts, clear PVC was no longer an option. Acrylic plastic was researched and found to be readily available in the dimensions needed for the chamber. Acrylic plastic is a very rigid material, but it is still ductile enough not to shatter from accidental impacts. The down side to acrylic plastic was its cost of twenty-eight dollars per foot. After some debate, acrylic plastic was selected as the chamber material.

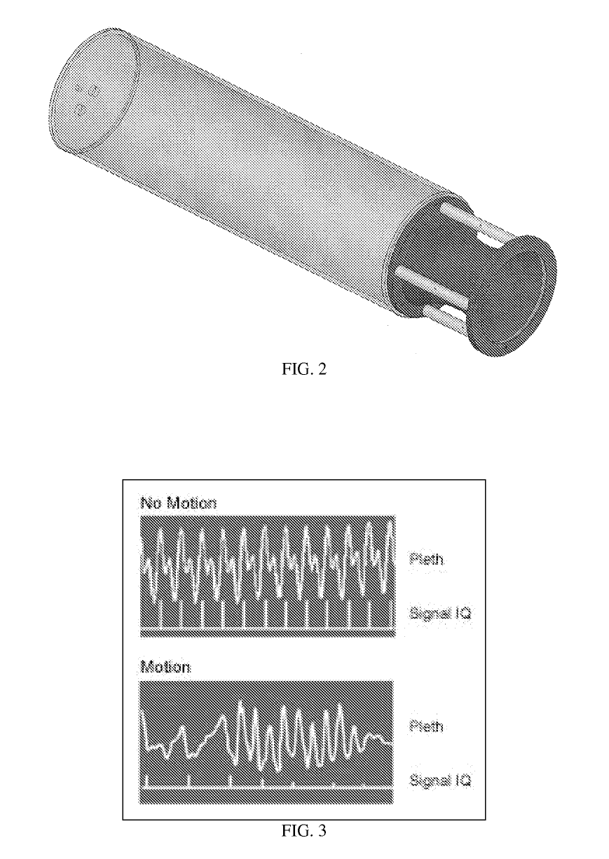

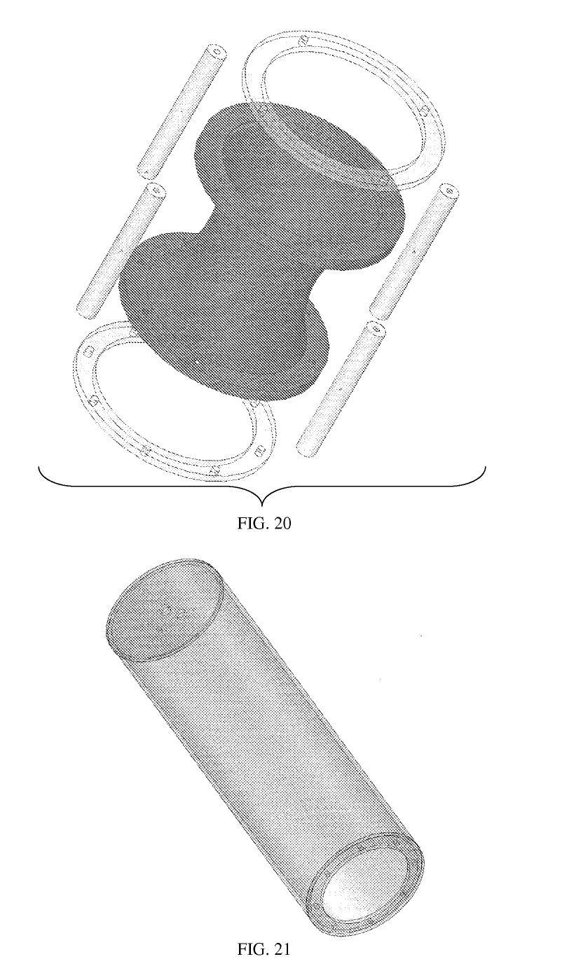

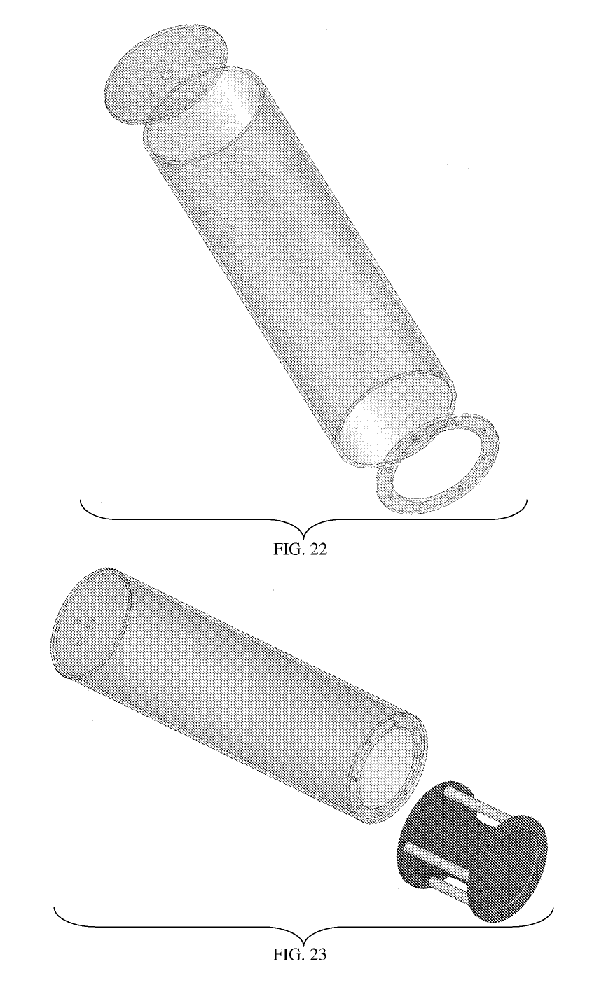

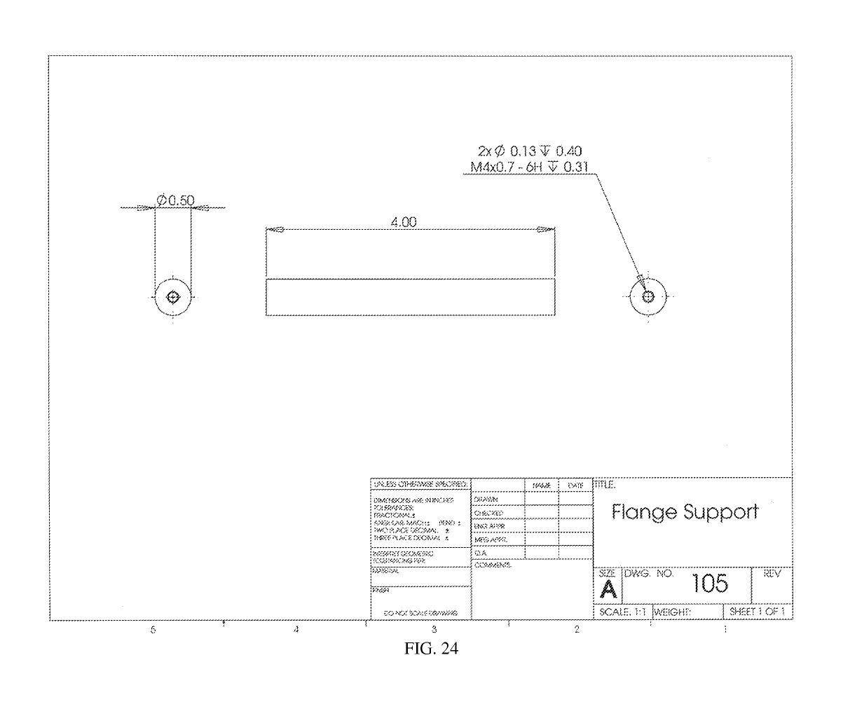

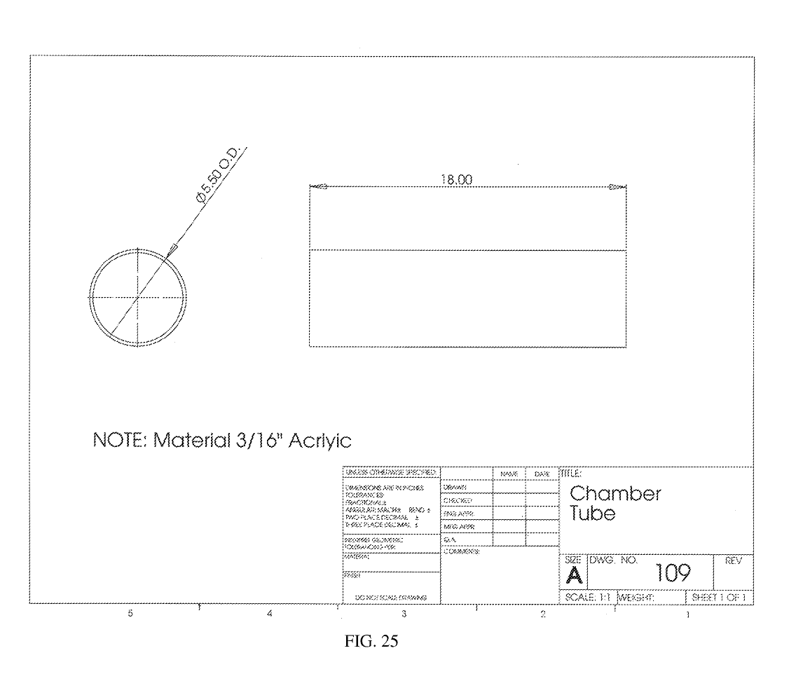

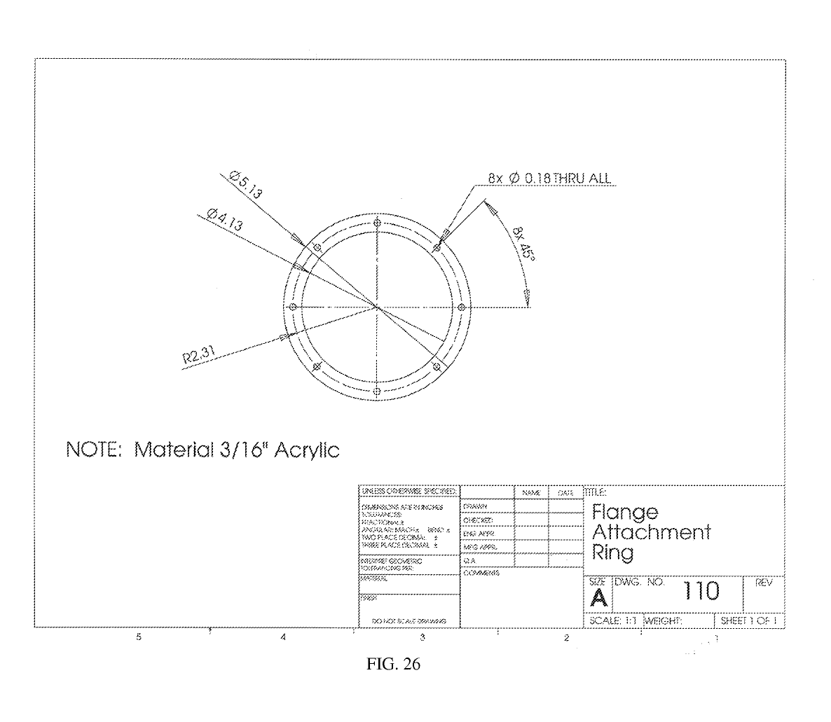

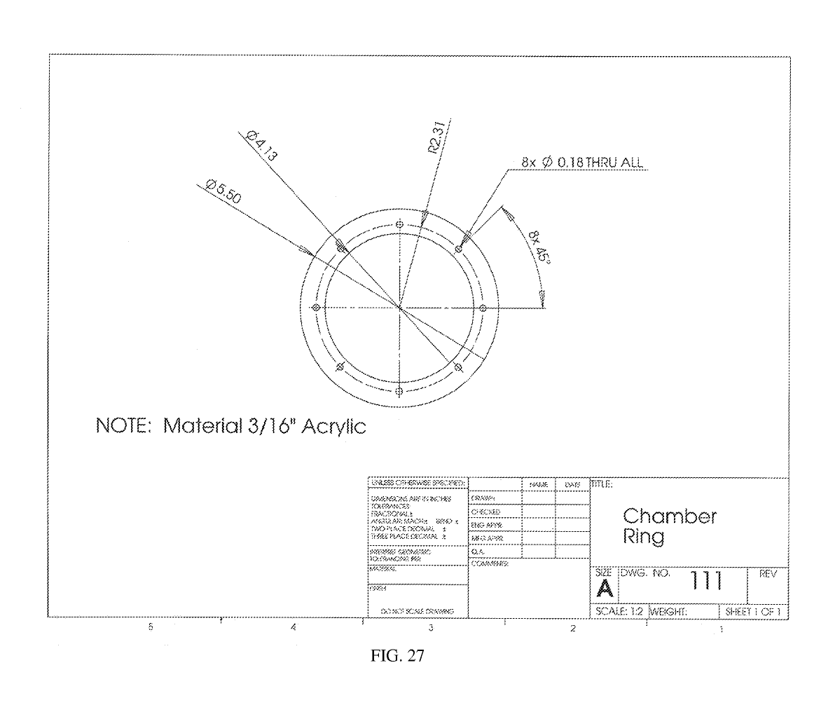

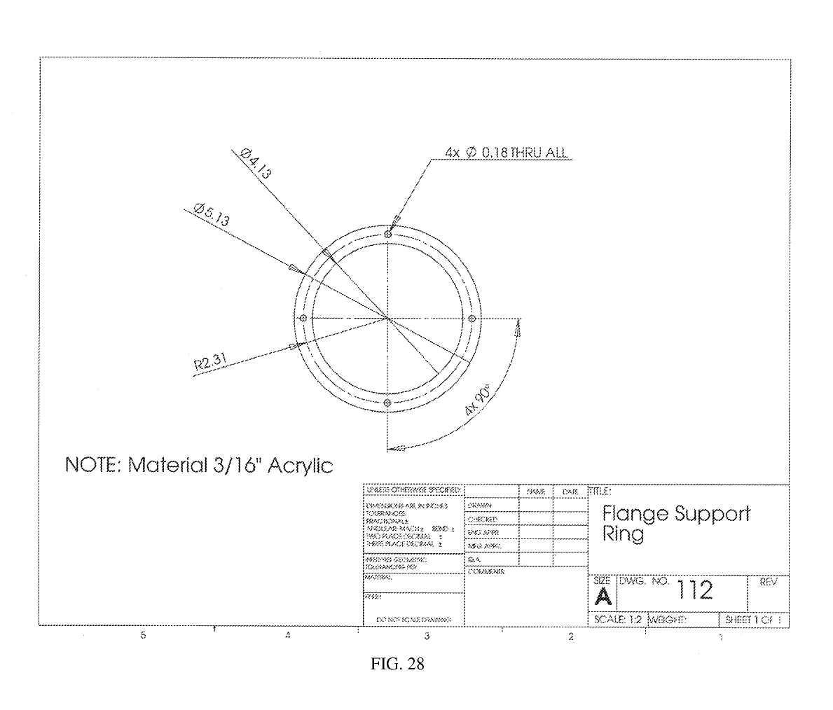

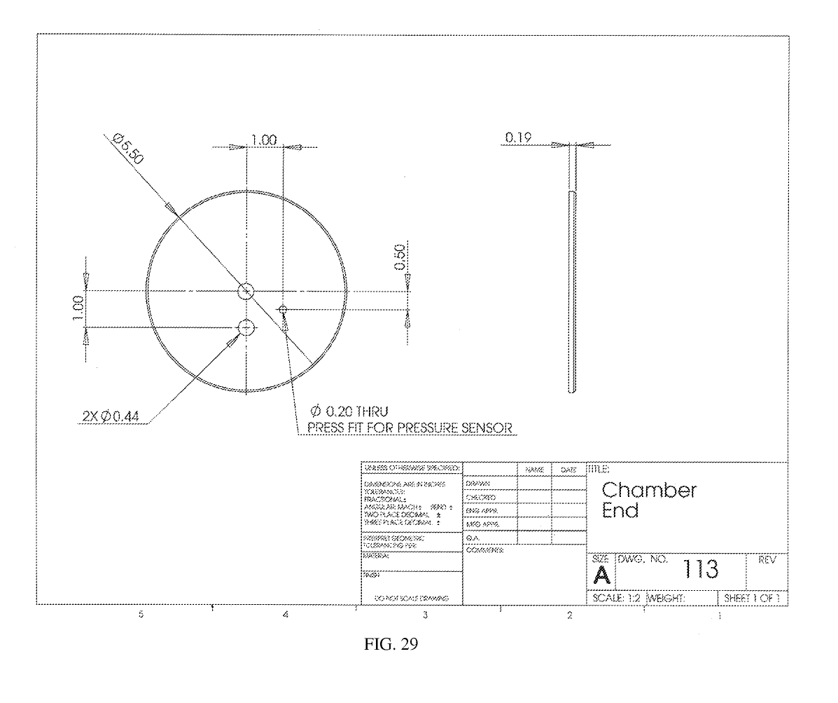

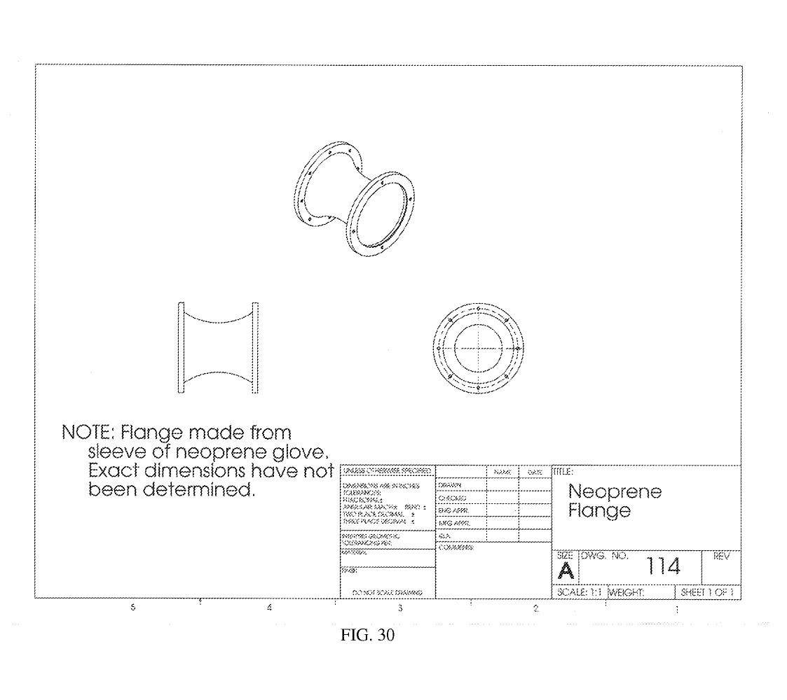

After contacting several companies, Patriot Plastics was selected to supply the acrylic plastic. Patriot Plastics is a Massachusetts company that supplies products that are customized to the buyer's requirements. A 3/16″×5½″×18″ tube of acrylic plastic was ordered along with a 6″×6″ flat stock sheet of acrylic plastic to seal the end of the cylinder. Additional plastic was needed to construct the seal around the flange. In FIG. 2, a computer draft of the chamber is displayed with all dimensions properly displayed. More detailed drawings of this chamber design located in FIGS. 19-30.

In order to bond the end cap and flange seals to the chamber cylinder, a bonding material needed to be selected. Perma Poxy is intended for use on hard plastics and has a curing time of five minutes. It creates an adhesive seal between two pieces of acrylic plastic but will not chemically combine the two together. Perma Poxy was tested on two scrap pieces of acrylic plastic and did not completely cure over the course of twelve hours. This could be contributed to the fact that it is a two part epoxy that requires even amounts of each part. A lack of hardener during the mixing process could have caused the epoxy to not cure. A representative from Patriot Plastics recommended using methalyne chloride to bond the acrylic plastic. Weld-On #4, which contains methalyne chloride, chemically bonds acrylic plastics together. Weld-On #4 was ordered from RPlastics, and was tested on scrap acrylic to test the effectiveness of the bonding agent. From testing, it was realized that the bond created was much greater then any adhesive medium could create. When applied, the Weld-On #4 begins to melt the acrylic and starts a bonding process immediately. After applying pressure for several minutes a bond is created that is sufficient enough to hold the pieces together. Over the next 48 hours the bond will continually increase to an equivalent strength close to a solid piece of acrylic. Concluding from the test results of the Perma Poxy and Weld-On #4, a decision was made to use the Weld-On #4 as the bonding agent for the sub-atmospheric chamber. Weld-On #4 was chosen as the bonding agent due to its strength after reacting with the acrylic and its aesthetic appearance of having no bonding medium remaining after curing.

B. Flange Design

The flange creates an air tight seal and at the same time does not impede venous return. To avoid constricting blood flow past the flange, the force being applied by the flange must be spread out across the area of the upper forearm. Several flange designs were considered using various materials. The first design used a modified latex dish washing glove with a strap that would tighten the glove to the forearm. Another option for the first design of the flange was to apply a plastic wrap and adhesive dressing around the dishwashing glove to create an air tight seal, which would apply minimal pressure. The plastic wrap adhesive dressing is used by the Vacuum Assisted Closure device to seal large wounds. Use of this dressing would reduce the amount of pressure needed by the strap to produce an air tight seal. The second used dry-suit extremity gaskets, either an arm gasket or ankle gasket depending on actual dimensions of the gasket. These gaskets provide a tight seal around the forearm not requiring the use of a load applying strap. The plastic wrap dressing may also be used to insure an air tight seal around the flange and forearm. The third design was to directly seal the chamber to the forearm using the plastic wrap adhesive dressing.

Each flange design can be attached to the chamber in several ways. One way is by pulling the flange medium over the chamber and securing it with a clamp. The other method is to create a flange seal that will clamp the flange between two rings of acrylic plastic. Each ring will have eight holes drilled in it; one ring will be secured to the chamber using Weld-On #4 and the other ring will be bolted to the first ring using 4 mm bolts with the flange clamped in between. This design can be seen in FIGS. 19-30.

The chamber was constructed using the rings because initial determinations were that the vacuum created would be so great that a simple clamp holding the flange medium on the chamber would not provide enough support to the flange. The flange medium used for initial testing was a latex dishwashing glove. When initially fitted, the modified latex glove conformed and sealed to the upper forearm very well. The latex dishwashing glove was chosen over the dry-suit seals due to the unavailability of the dry-suit parts and over the plastic wrap adhesive due to the lack of support provided by the plastic wrap.

The latex flange was tested for blood flow restriction and discomfort in order to determine acceptable pressure which the flange could apply before restricting blood flow. The results from these tests are displayed in Table 1. These tests were conducted on three subjects. The blood perfusion during the tests was measured using the Radical™ pulse oximeter supplied by the Masimo® Corporation. During testing, the pulse oximeter lead wire was run through the flange to test for comfort and to ensure accuracy of results. None of the test subjects complained of discomfort or showed signs of blood loss to the forearm or hand.

Table 1-Flange Testing Data from Pulse Oximeter

TABLE 1
Flange Testing Data from Pulse Oximeter

| Time (minutes) | Beats Per Minute (BPM) | Saturation |
|---|---|---|
| Subject 1 Without Flange | | |
| 1 | 74 | 98 |
| 2 | 70 | 98 |
| 3 | 80 | 97 |
| 4 | 69 | 98 |
| 5 | 74 | 98 |
| Subject 1 With Flange | | |
| 1 | 71 | 98 |
| 2 | 72 | 98 |
| 3 | 74 | 98 |
| 4 | 72 | 99 |
| 5 | 74 | 98 |
| Subject 2 Without Flange | | |
| 1 | 82 | 97 |
| 2 | 80 | 97 |
| 3 | 79 | 98 |
| 4 | 84 | 97 |
| 5 | 75 | 96 |
| Subject 2 With Flange | | |
| 1 | 82 | 97 |
| 2 | 81 | 97 |
| 3 | 84 | 96 |
| 4 | 82 | 95 |
| 5 | 79 | 97 |
| Subject 3 Without Flange | | |
| 1 | 68 | 99 |
| 2 | 72 | 99 |
| 3 | 68 | 99 |
| 4 | 66 | 98 |
| 5 | 70 | 99 |
| Subject 3 With Flange | | |
| 1 | 65 | 100 |
| 2 | 76 | 99 |
| 3 | 74 | 100 |
| 4 | 81 | 98 |
| 5 | 69 | 98 |

From this test it was realized that the pressure applied by the latex would be of an appropriate quantity for the flange.

Once the sub-atmospheric chamber and vacuum system was designed and assembled the flange and flange seal were tested to see if an appropriate seal was formed. Initially the flange was attached to the chamber at the flange seal and would descend into the chamber. When a vacuum was applied, air would be drawn immediately into the chamber between the forearm and flange, lifting the flange off of the forearm. To try to fix this problem the flange was pulled out of the chamber and clamped to the forearm. Once this was performed and a vacuum was applied the flange would immediately be sucked into the chamber, with similarity to a balloon being inflated. Several problems were realized with this set up. The pressure needed to hold the flange to the forearm to keep it from getting sucked into the chamber. Blood flow restriction would increase as the latex flange began to balloon, applying additional pressure to the forearm. After analyzing the reaction of the latex flange when a force is applied relative to the vacuum, several design changes were made.

When a vacuum is applied to the sealed flange, the flange immediately begins to be drawn into the chamber. In order to stop this occurrence a support needed to be designed which would keep the flange from being deformed and drawn into the chamber. At the same time the new flange design would not apply any forces to the forearm which could restrict blood flow. The occurrence of the flange creating a tighter seal when held back from the applied vacuum was taken into consideration when developing the new flange design. The flange design shown in FIGS. 19-30 incorporates an acrylic ring identical to the ring used to clamp the flange to the chamber and four, 4-inch aluminum spacers. The flange will wrap around the acrylic ring in similar fashion to the acrylic ring used in the flange seal. The aluminum spacers are inserted between the rings stretching the flange medium, and are bolted to the chamber and acrylic ring using 4 mm bolts. With this new design the flange applies minimal pressure to the forearm until a vacuum is applied. The applied vacuum will begin to suck the flange into the chamber as seen in previous tests but the flange is restricted from being pulled into the chamber by the supports. Also, the slight amount that is pulled into the chamber produces the sealing pressure to the forearm that is necessary to create an air tight seal.

After testing this design it was realized that the flange material would need to be changed. Increasing the thickness of the flange material would reduce the amount of flange that deformed under vacuum and would keep the flange from tearing due to applied pressure and normal wear and tear. Also taken into consideration when choosing a new flange material is how the material would react with the skin of a patient. Latex although readily available is no longer used in a hospital environment due to the increased numbers of allergic reactions patients have with the material. Taking these factors into consideration, neoprene was chosen for the new flange material. Neoprene gloves made for industrial applications are readily available in many sizes and thicknesses. Sizes 7, 8, and 9 were ordered from the McMaster catalog in a thickness of 30 mils. After receiving the neoprene gloves and performing several tests on functionality and sizing it was determined that the neoprene gloves were ideal for the current flange design. After modifying the neoprene gloves a size range was determined. Size 7 would be ideal for a person in the 120-145 lb weight range, size 8 would be ideal for a person in the 145-170 lb weight range, and size 9 would be ideal for a person in the 170 lb and above weight range.

The selected flange configuration applied essentially no pressure (i.e., no pressure beyond the minimal pressure resulting from contact of the surfaces) to the extremity at atmospheric pressure, but when vacuum is applied the sealing pressure of the flange increases with an increase in vacuum creating an air tight seal. Then, when vacuum is reduced it returns back to its static state. As a result, venous return is not impaired by the flange during periods of atmospheric pressure, without the need for separate control of flange pressure.

C. Vacuum System

The first system that was devised had a vacuum pump continuously running with an electrical solenoid valve in between the vacuum pump and the chamber which would control the vacuum pressure to the chamber. This system had either the same solenoid that was controlling the vacuum or another solenoid valve open the chamber to atmosphere to equalize the pressure in the chamber back to atmospheric pressure.

While both the solenoid valves and the vacuum pumps were researched simultaneously, the vacuum pump needed to be selected first. Judging by the pressure requirements set forth in the functional requirements, a few fluid calculations were conducted in order to determine the specifications required for the vacuum pump. The calculations seen below were used to figure out how much volume would need to be evacuated to get the 75 mmHg drop in pressure, which is 3″ Hg, or 1.5 psi, or 10 kPa, or 40″ H₂0, or about 10% of atmospheric pressure.

$$PV = mRT$$

$${m}_{1} = \frac{{P}_{1} V}{RT} = \frac{( 101.3 k Pa ) ( 0.00566 {m}^{3} )}{( 286.9 J \text{/} kg K ) ( 293 K )} = 0.00682 kg$$

$$\begin{matrix}{m}_{2} = \frac{{P}_{2} V}{RT} = \frac{( 91.3 k Pa ) ( 0.00566 {m}^{3} )}{( 286.9 J \text{/} kg K ) ( 293 K )} = 0.00615 kg \\ {m}_{1} - {m}_{2} = 0.00682 kg - 0.00615 kg = 6.734 \times {10}^{- 4} kg \\ \begin{matrix}V = \frac{mRT}{P} \\ = \frac{( 6.734 \times {10 }^{- 4} kg ) ( 286.9 J \text{/} kg K ) ( 293 K )}{( 101.3 k Pa )} \\ = 5.591 \times {10}^{- 4} {m}^{3} \\ = 0.0197 {ft}^{3}\end{matrix}\end{matrix}$$

From the calculations, it can be seen that the 10% drop in pressure resulted in an approximate 10% volume evacuation since the chamber holds 0.2 ft³. To simplify further calculations, the volume needed to be evacuated will be rounded to 0.02 ft³.

The next set of calculations conducted was used to determine the volume flow rate required from the vacuum pump. These calculations can be seen below.

$$Q = \frac{0.02 {ft}^{3}}{0.1 sec} ( \frac{60 sec}{1 min} ) = 12 {ft}^{3} \text{/} min = 12 cfm$$

$$Q = \frac{0.02 {ft}^{3}}{0.05 sec} ( \frac{60 sec}{1 min} ) = 24 {ft}^{3} \text{/} min = 24 cfm$$

$$Q = \frac{0.02 {ft}^{3}}{0.01 sec} ( \frac{60 sec}{1 min} ) = 120 {ft}^{3} \text{/} min = 120 cfm$$

To get the pressure drop in 0.1 seconds, the vacuum pump needed to have a flow rate of 12 cubic feet per minute. The ratio is inversely proportional, so to divide the time in half means the flow rate would have to be twice as much. Since finding a practical vacuum pump with over 12 cfm of air flow is difficult and a change in pressure in under 0.1 seconds may be too harsh on a person's arm, 12 cfm was chosen to be the volume flow rate of the vacuum pump. Also, a time of under 0.1 seconds could be strenuous but a time of over 0.1 seconds would be too slow to synchronize with a person's pulse.

Once the volume flow rate and the ultimate vacuum pressure were known, a vacuum pump could be located for this application. Other vacuum pumps were researched and a few companies with vacuum products that could be applied to this design were found.

The vacuum pumps that were originally considered were either regenerative blowers or rotary vanes. These pumps were eventually ruled out because of different constraints within the design. The regenerative blowers and rotary vanes were loud, large, and expensive which were properties not desired in this design. The vacuum pumps that seemed appealing for this design were venturi style vacuum pumps.

Venturi vacuum pumps are small vacuum generators that contain no moving parts and only require a compressed air source to operate. This style of vacuum generator was originally chosen but it was determined that it might be more expensive because of the need for a compressed air source. The JF-300, manufactured by Vaccon, was chosen for the design. This vacuum pump was chosen because it generated near instantaneous vacuum and could be used in a pulsed application, meaning the solenoid valve could be used on the compressed air source to turn the vacuum pump on and off instead of having the vacuum continuously run. This solved a major problem with the solenoid selection. This particular vacuum pump had ⅜ inch female NPT connections on all the ports, had a high flow silencer to dampen the sound, but was specified to run at 80 psi which was higher than typical hospital wall air sources can reach. However, the amount of pressure at the inlet from the compressed air source is directly related to how much vacuum is provided at the vacuum inlet. So, since the vacuum was capable of a 10″ Hg drop in pressure at 80 psi and the design only required a 3″ Hg drop, then the pressure from the compressed air source did not have to be nearly as high as 80 psi to reach 3″ Hg vacuum.

The design for the vacuum system was slightly changed now that the vacuum pump was going to be controlled by a solenoid valve on the compressed air line. The new design called for two solenoid valves with one controlling solenoid valve to turn on and off the vacuum pump and one equalizing solenoid valve to equalize the pressure in the chamber back to atmospheric pressure.

The selection process for the controlling solenoid valve was simplified once it was determined that the solenoid valve would be placed on the compressed air line and not between the vacuum pump and the chamber. This meant the solenoid valve would be experiencing up to 80 psi of compressed air and not just 1.5 psi of vacuum pressure. This increase in differential pressure and the decrease in orifice size compared to other vacuum pumps meant that the solenoid valve could open more easily and more quickly.

After researching many different types of solenoid valves, the manufacturer ASCO was found that had the appropriate valves for this design. While the original plan was to have the solenoids open by a 12VDC coil, the lack of these in stock and the need for a power supply to operate them led the design to use 120VAC coils. By using 120VAC coils, the solenoid valves could be operated with normal wall outlet power. The controlling valve that was chosen for this design was the 8210G001, which has a 120VAC coil and a normally closed ⅜ inch valve. The equalizing valve that was chosen for this design was the 8262G90, which has a 120VAC coil but a ¼ inch normally closed valve. Both these valves operated within the proper pressure range required and had the correct port and valve sizes.

D. Pulse Sensing

Pulse oximetry was selected as a technique for sensing pulse because such a technique has at least two advantages over the more traditional pressure sensor: first, pulse oximeters sense pulse by monitoring blood flow, so they are less susceptible to movement artifacts than are pressure sensors, which typically respond to, e.g., gross muscle movement as well as to the pulse, thereby rendering the pulse measurement meaningless. Second, a pulse oximeter can detect pulses even when the subject is in a low-perfusion state. Pressure sensors can sense an arterial pulse only when the pulse strength exceeds a fairly high threshold. A pulse oximeter, in contrast, can sense a pulse even when the pulse cannot be palpitated.

A Radical™ pulse oximeter was obtained from Masimo® Corporation. The serial port of the Radical pulse oximeter outputs two analog signals. The user can select between 0V, 1V, pulse rate, pleth waveform, oxygen saturation percentage, and Signal IQ as the output for either channel. The information contained in these signals is transmitted through a linear range of 0V to 1V.

Initially, system control was going to be achieved through analysis of the pleth waveform output through the serial port of the device. The sub-atmospheric pressure was to be produced at anytime the pleth waveform had a positive slope. Upon testing, it was determined that the pleth waveform was sometimes unreliable. Anytime finger motion occurred or pressure was applied to the finger sensor an erratic waveform resulted. In addition, the standard waveform produces two segments of positive slope for each pulse. Also, the segment of the waveform for which the lower pressure is necessary occurs in only a small fraction of the total pulse.

Through analysis of the other output modes, it was determined that the Signal IQ output was better suited to the needs of this project. This function determines the peak of each pulse. When each peak occurs a pulse is sent through the analog channel of the serial port. Comparison between the pleth waveform and Signal IQ is shown below in FIG. 3 (Masimo).

The Signal IQ has many advantages. First, output continues throughout a low perfusion state. Under such conditions, the pulse intensity decreased from 1V to as low as 100 mV. As the output already required signal conditioning for input into the microcontroller, the circuit will now include a voltage comparator to compensate for the decreased intensity. This setup will allow for the device to operate under low tissue perfusion conditions. Secondly, the pulse output will act as a trigger within the microcontroller. This allows for far less complicated programming.

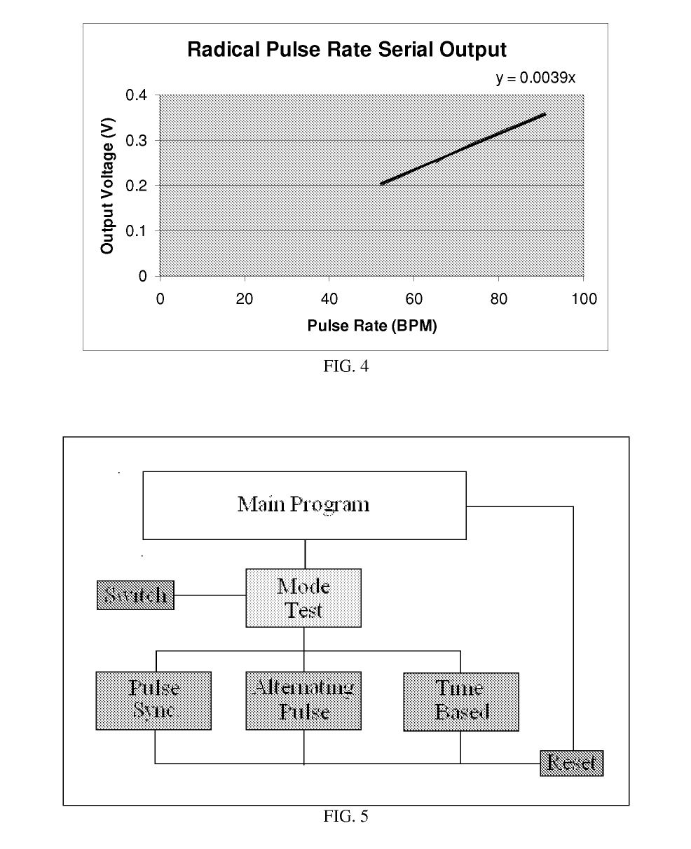

The second analog channel is used to output the pulse rate from the Radical. This signal allows for calculation of cycle times within the programming. Using Microsoft Excel the relationship between the pulse rate and voltage output was determined as shown in FIG. 4.

Output voltage varies linearly from 0V with a pulse rate of 0 beats per minute (BPM) to 1V. Based on the data obtained, the maximum voltage output would occur at 255 BPM. This output is interfaced to the microcontroller through the A/D converter.

E. Programming

As this project is designed to collect data proving a positive correlation between sub-atmospheric pressure and peripheral blood circulation, no cycling method or duration is known to be better than any other. For this reason multiple modes have been programmed for the system. Three pressure control modules, a mode testing module, LCD initializing module, atmosphere and vacuum testing modules, and modules for reading and displaying pressure are contained with the complete program.

The main program is simply a shell running in a constant loop. After initializing the LCD, the program waits for a pulse signal from the pulse oximeter to verify that the device is properly setup. At this time “Wait for Pulse” is displayed on the LCD screen. After receiving the signal, the mode test program is called. Based on the result, the program will then call on one of the three modes. If no mode has been selected, the program will continue to loop with every pulse signal while displaying “Select Mode.” A simple flow chart of the main program is given in FIG. 5.

The mode testing program uses a six-position switch that is connected to four input pins on the microcontroller. The switch has been configured to move between four of the six available positions. Based on the switch position, the function within the module generates one of four values: 0, 1, 2, and 3. These values correspond to the If-Else statements in the main program. This module then exits to the main program returning the proper mode. To change to a different mode the user must move the switch to the desired position and then press the reset button. Both of the buttons are located next to the LCD screen. The reset button is necessary because the mode testing module is only called from the main program.

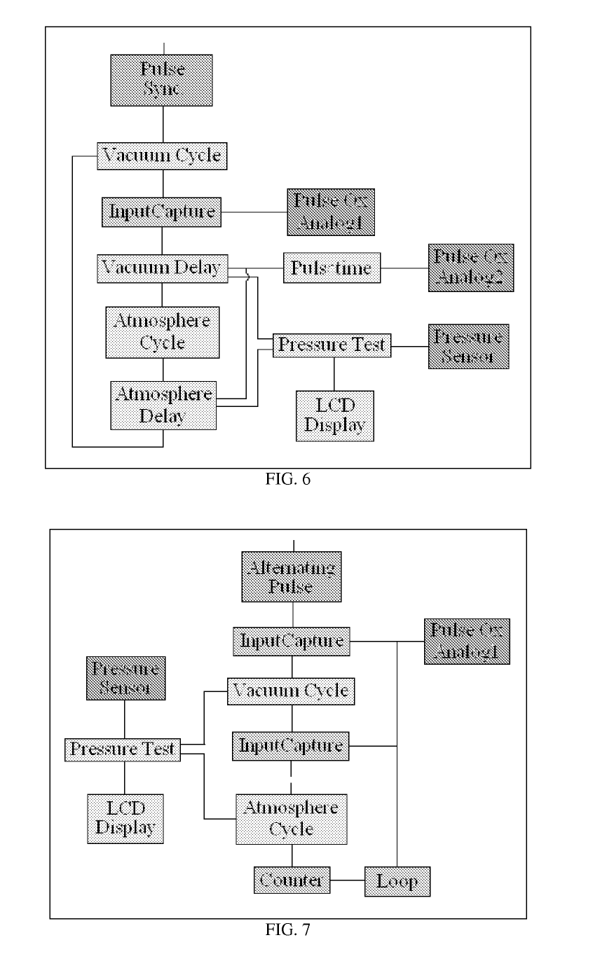

The first pressure control module is designed to be synchronized with every pulse. Like the main program and the other pressure control modules, this module is a continuous loop. The module initializes the vacuum cycle by opening the compressed air solenoid and closing the chamber solenoid. Then, the vacuum error testing program is called and run continuously until the pulse signal is received. The pulse rate is calculated from the analog input from the pulse oximeter. Based on the pulse rate, a delay is entered into for a percentage of that time. At the end of the delay the solenoid states are reversed, and the chamber returns to atmospheric pressure. At this time another delay is initialized while the atmosphere error testing program is run. The flow chart of the module is shown in FIG. 6.

This is the most complicated of all the modules, due to the overlapping operations. The order of operations was determined to mitigate the number of potential conflicts. The vacuum cycle is currently set to continue for twenty percent of the pulse rate after the Signal IQ input. The atmosphere cycle is set to continue for half of the entire cycle. The sum of the two delays must be less than the total time between pulses to ensure that the pulse input for the next cycle is not missed. The current settings reserve 30 percent of the cycle time for delays associated with the LCD screen and other general operations. LCD delays are necessary because the Com3 port is dedicated to the “InputCapture” command as soon as it is called. The LCD screen requires approximately 0.5 ms per character. If the LCD queue has not been emptied prior to the “InputCapture” command, errors occur in the display. The remaining reserved cycle time is used to anticipate the next pulse signal. This is necessary due to the fluctuation in time between pulse signals that occur in both steady and erratic pulse rates.

The second pressure control module is much simpler than the first. This module is designed to sustain vacuum pressure for the entire duration between pulses and then return to atmospheric pressure for multiple pulses using two loop statements. At the beginning of the primary loop a counter is set to some value. After receiving the pulse oximeter signal, the vacuum cycle and vacuum testing programs are initialized. The module then enters a secondary loop. After the next pulse oximeter signal, the solenoid states are reversed so that pressure returns to atmosphere. Also, the atmosphere error testing module is called. Pressure will remain at atmosphere throughout the remainder of this loop. The counter is decremented, and this secondary loop continues until the counter reaches zero. After exiting this loop, the primary loop then repeats. The value set for the counter is currently three. This module can be seen in FIG. 7.

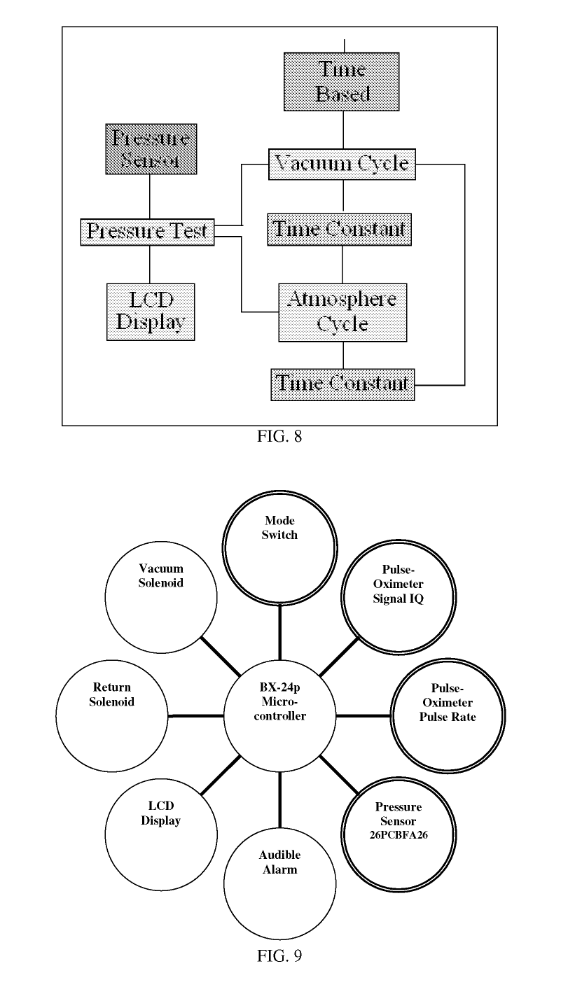

The final pressure control program is based on time and is independent of the pulse oximeter as shown in FIG. 8. Procedure for the vacuum states and test modules is the same as the other control modules. Time for each cycle is preset and regulated by two delays. The vacuum cycle is currently set for 5 seconds, and the atmosphere cycle for 15 seconds.

The LCD screen displays the mode name as well as the chamber vacuum pressure. The vacuum pressure is calculated using an analog input voltage and displayed in mmHg. In addition, two error programs have been written to make sure that the vacuum system is functioning properly. The first program checks the pressure during the vacuum cycle. The acceptable vacuum range is currently 25 to 150 mmHg. If the vacuum is lower than 25 mmHg, “Error1” is displayed. If the vacuum pressure exceeds 150 mmHg, “Error2” is displayed. The second error program tests the vacuum pressure during the atmosphere cycle. If the vacuum pressure is not below 25 mmHg, “Error3” is displayed. Anytime that an error is encountered an audible alarm is sounded. All three of the programs with comments are appended to the specification.

F. System Control

A microcontroller based system was decided to be used versus an analog circuit. Microcontrollers offer an advantage in ease of use and flexibility in modifying the program. Multiple microcontrollers were researched to decide the best choice for the system. The factors that were important for selection were frequency, memory, input/output ports, price, and analog inputs. The BasicX-24P was chosen to best fit this project. Table 2 shows some specifications of the BasicX-24P.

Table 2-BasicX-24P Specifications

TABLE 2
BasicX-24P Specifications

| | |
|---|---|
| Speed | 83,000 Basic instructions per second |
| EEPROM | 32K bytes (User program and data storage) |
| Max program length | 8000+ lines of Basic code |
| RAM | 400 bytes |
| Available I/O pins | 21 (16 standard + 2 serial only + 3 accessed |
| | outside standard dip pin area) |
| Analog Inputs (ADCs) | 8 (8 of the 16 standard I/O pins can individually |
| | function as 10 bit ADCs or standard digital I/Os |
| | or a mixture of both) |
| Serial I/O speed | 1200-460.8K Baud |
| Floating point math | 32 bit × 32 bit floating point math built-in |
| Programming interface | High speed Serial |
| Physical Package | 24 pin DIP module |

One of the most important characteristics of the BasicX-24P is eight 10-bit analog to digital converters capable of 6,000 samples per second. These are used to take an analog input from the pressure sensor as well as the Pulse Rate from the Radical pulse oximeter.

A block diagram of the electrical circuits used to control the system is shown in FIG. 9. The BasicX-24p is used as the heart of the system and has 4 inputs and 4 outputs. The inputs to the microcontroller are indicated with double outlines in FIG. 9, and the outputs are indicated with single outlines. The mode switch is connected to 4 inputs on the BX-24p, and it is used to switch between the 3 different modes and an off position. The Signal IQ and Pulse Rate inputs are from the serial connection with the Radical pulse oximeter. The 26PCBFA26 pressure sensor is an analog input to the microcontroller placed inside of the chamber. If the pressure is out of the specified range then an audible alarm will sound. On the LCD display, the pressure will be shown from the sensor, and the chosen mode will be displayed. The vacuum solenoid and return solenoid are opened and closed through driver circuits with inputs from the microcontroller.

(1) Input Signal Conditioning Circuits

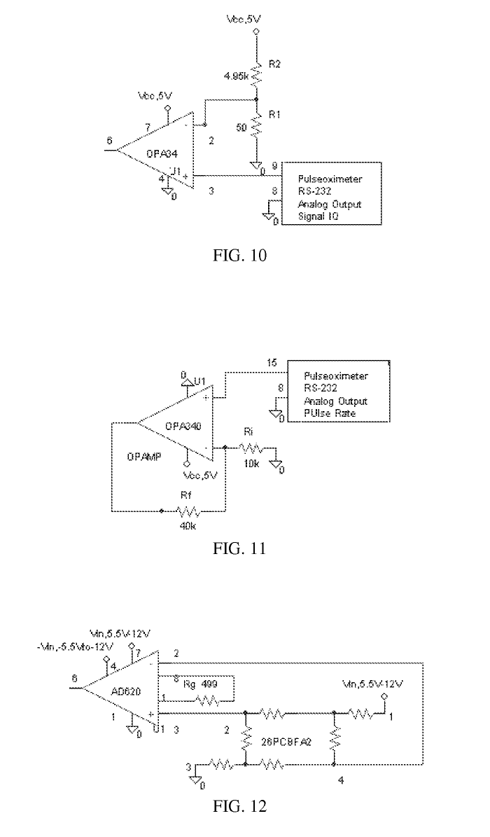

As previously mentioned the Radical pulse oximeter outputs a 1V pulse, referred to as the Signal IQ. This pulse signal must be amplified to 5V for use as an input for the microcontroller. Under low perfusion conditions the pulse must be amplified from 100 mV to 5V. To condition this range of voltages a voltage comparator circuit was selected. The circuit uses a single-supply +5V OPA340 operational amplifier. The reference voltage is produced by a voltage-divider as seen connected to the (−) input to the op-amp. This voltage has been chosen to be 50 mV, to satisfy the range of the Signal IQ output on the Radical pulse oximeter from 100 mV to 5V. Both voltages are supplied by the microcontroller 5V output (pin 21). Anytime the pulse is greater than the reference voltage, the op-amp will be in saturation, and 4.99V (high input) will be supplied to the microcontroller (pin 20). When the pulse is not greater than the reference voltage then op-amp will output 10 mV which is in the microcontroller's low input range. This circuit is seen in FIG. 10 with the theoretical resistor values.

The circuit seen in FIG. 10 had an actual R2 resistor value of 4.866 kΩ, and R1 value of 48Ω. Using the voltage divider equation seen below as equation 1, and the calculation that follows, the actual reference voltage was found to be 48.9 mV. This was tested and found to be accurate by setting the R2 and R1 resistors in series and applying 5V to the R2 resistor. The voltage across the R1 resistor with respect to ground was measured to be 48.9 mV using a digital multi-meter.

$${V}_{R 1} = \frac{R 1}{( R 1 + R 2 )} * {V}_{in} \\ {V}_{R 1} = \frac{48 Ω}{( 48 Ω + 4.866 * {10}^{3} Ω )} * 5 V \\ {V}_{R 1} = 48.9 mV \qquad ( 1 )$$

An oscilloscope was attached to the output of the OPA340 op-amp in FIG. 10 to test the circuit. When the Signal IQ from the RS-232 serial output was attached to the (+) input of the op-amp, 5V pulses were seen on the oscilloscope. Every time a pulse from was seen on the Radical pulse-oximeter, a 5V pulse was seen on the oscilloscope.

The Pulse Rate output from the Radical pulse-oximeter outputs a pulse between 0V and 1V that is proportional to the beats/minute displayed on the pulse-oximeter. This voltage must be amplified between 0V and 5V to be accepted by the analog inputs of the BX-24p microcontroller. This is a gain of 5. The negative feedback circuit seen in FIG. 11 was developed to amplify the necessary gain.

The gain is set by equation 2 seen below. The value of the Ri resistor was chosen to be 10 kΩ. Through the calculation seen below, the Rf resistor was calculated to be 40 kΩ.

$$Gain = 1 + \frac{Rf}{Ri} \\ 5 = 1 + \frac{Rf}{10 k Ω} \\ 40 k Ω = Rf \qquad ( 2 )$$

The circuit was built using 4-10 kΩ resistors in series to act as Rf, and 1-10 kΩ resistor as Ri. The pulse-oximeter was attached to a finger to give a live pulse and allow for an output from the pulse-oximeter. The output voltage from the pulse-oximeter was measured to be 0.39V using a digital multi-meter. The output voltage from the OPA340 op-amp was measured to be 1.95V. Dividing the output voltage of the OPA340 by the output voltage of the pulse-oximeter gives a gain of 5.

A differential voltage between 0 mV and 50 mV is outputted from the 26PCBFA26 pressure sensor. The pressure sensor has a typical supply voltage of 10V. To be accepted by the analog inputs from the microcontroller the output voltage must be amplified to a range from 0V to 5V, a gain of 100 from the 0 mV to 50 mV sensor output. This is done using the AD620 instrumentation amplifier, in the circuit seen in FIG. 12.

The gain for the amplification of the differential voltage is set by the Rg resistor on the AD620. This is calculated using equation 3 in the calculation below. The value of Rg was calculated to be 499Ω.

$$Gain = ( 1 + \frac{49.4 k Ω}{Rg} ) \\ 100 = ( 1 + \frac{49.4 k Ω}{Rg} ) \\ Rg = 499 Ω \qquad ( 3 )$$

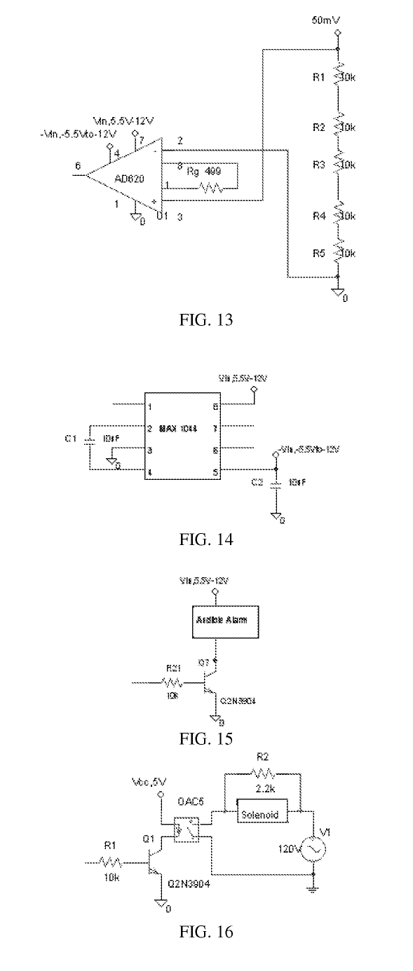

The circuit seen in FIG. 13 was built to test the gain of the pressure sensor signal conditioning circuitry. The differential input voltage to the circuit was set by changing the position of the (+) input on the series resistors divider. The maximum input voltage is 50 mV, and the minimum input voltage is 0 mV. These values were chosen because they represent the range of the 26PCBFA26. The results of these tests, as well as the calculated gains are shown in Table 3.

Table 3-Test Results of Pressure Sensor Signal Conditioning Circuit

TABLE 3
Test Results of Pressure Sensor Signal Conditioning Circuit

| Trial | Vdiff (mV) | Vout (V) | Gain |
|---|---|---|---|
| 1 | 50.0 | 4.94 | 98.8 |
| 2 | 40.2 | 3.96 | 98.5 |
| 3 | 30.5 | 2.99 | 98.0 |
| 4 | 19.9 | 1.94 | 97.5 |
| 5 | 9.9 | 0.95 | 96.0 |
| 6 | 00.2 | −1.5 | — |

The gain was adjusted to be approximately 100 in the program inside of the microcontroller for the range between 0 mV and 20 mV from the pressure sensor. This is the range that will be used during use of the entire system. When this was done the values in Table 4 were displayed on the LCD screen from the microcontroller when the pressure sensor signal conditioning circuit was attached to pin 13 of the microcontroller.

Table 4-Test Results of Pressure Sensor Signal Conditioning Circuit Through LCD Display

TABLE 4
Test Results of Pressure Sensor Signal Conditioning
Circuit through LCD Display

| Trial | Vdiff (mV) | Display | Gain |
|---|---|---|---|
| 1 | 15.3 | 1.52 V | 99.3 |
| 2 | 9.67 | 0.97 V | 100.3 |
| 3 | 5.36 | 0.54 V | 100.7 |

The pressure sensor was tested for accuracy from a range of 0 psi to 5 psi. The pressure sensor was attached to the output of an air regulator, and the value of air pressure through the regulator was adjusted. The pressure sensor was first tested directly, reading the differential output between the voltages with a digital multi-meter. This data is shown in Table 5. For this test run the sensor was powered by a 10V supply, the typical supply voltage for the sensor. The pressure sensor was then tested with a supply voltage of 8.95V from the microcontroller board. This data is shown in Table 6. The third trial using the IC-11 air regulator used the pressure sensor signal conditioning circuitry and shows the output from that, Table 7.

Table 5-Pressure Sensor Test Results (10V Supply)

TABLE 5
Pressure Sensor Test Results (10 V supply)

| | IC-11 Air Regulator | Pressure Sensor |
|---|---|---|
| Trial | (psi) | (mV) |
| 1 | 0 | 0 |
| 2 | 1.25 | 12.2 |
| 3 | 2.5 | 26.0 |
| 4 | 3.75 | 36.9 |
| 5 | 5 | 50.0 |

Table 6-Pressure Sensor Test Results (8.95V Supply)

TABLE 6
Pressure Sensor Test Results (8.95 V supply)

| | IC-11 Air Regulator | Pressure Sensor |
|---|---|---|
| Trial | (psi) | (mV) |
| 1 | 0 | 0 |
| 2 | 1.25 | 16.5 |
| 3 | 2.5 | 25.7 |
| 4 | 3.75 | 34.9 |
| 5 | 5 | 45.0 |

Table 7-Pressure Sensor Signal Conditioning Circuitry Test Results (8.95V Supply)

TABLE 7
Pressure Sensor Signal Conditioning Circuitry
Test Results (8.95 V supply)

| | IC-11 Air Regulator | AD620 output |
|---|---|---|
| Trial | (psi) | (V) |
| 1 | 0 | 0 |
| 2 | 1.25 | 1.93 |
| 3 | 2.5 | 3.02 |
| 4 | 3.75 | 4.00 |
| 5 | 5 | 5.21 |

The AD620 instrumentation amplifier requires both a positive and negative supply voltage. The positive supply voltage came from the microcontroller board at 8.9V. To create the negative supply voltage, the MAX1044 switched-capacitor voltage converter was used. The circuit seen in FIG. 14 was developed around the MAX1044.

When 4.85V was attached to the input supply voltage pin on the MAX1044 the negative output voltage was measured to be −4.85V using a digital multi-meter. This is a gain of −1. 8.9V was then attached to the input supply voltage, and a reading of −8.9V was received.

(2) Output Driver Circuits

The audible alarm circuit was developed using the 2N3904 transistor using a common emitter circuit seen in FIG. 15. The audible alarm was placed between the 8.9V output from the microcontroller and the collector of the transistor. A 10 kΩ resistor was placed to divide the voltage drop across the base of the transistor from the output of the microcontroller. The circuit was tested by applying 5V to the 10 kΩ resistor. When this was done a buzzing sound was heard from the audible alarm.

A circuit was developed to open and close the 120VAC solenoids using the 5V output of the microcontroller. This utilizes the OAC5 5V to 120VAC solid state relay. The input to the relay is from 3-8V. As a voltage is applied to the base of the resistor a current flows from the common to the emitter and through the input of the relay. This closes the 120VAC loop on the other side of the relay and opens the solenoid. The relay requires a minimum current of 50 mA through the output of the relay, and a current of only 16 mA is required to hold the solenoid. For this reason, a 2.2 kΩ 10 Watt resistor is placed in parallel with the solenoid. This ensures that at least 55 mA will flow through the R2 resistor and the relay whenever the circuit is closed.

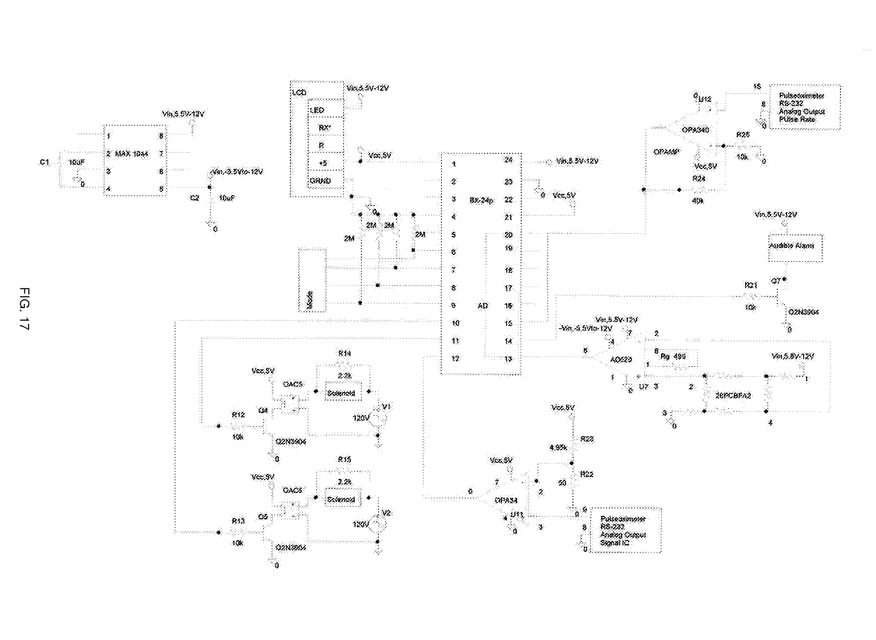

Using the circuit displayed in FIG. 16, the solenoid was heard to click signifying that it was opening when 5V was applied to the R1 10 kΩ resistor. Two identical circuits were built for using the complete system. One controls the vacuum solenoid, and the other controls the return to atmosphere solenoid.

(3) Complete Circuit Schematic

FIG. 17 displays the complete schematic for the BasicX-24p microcontroller based system. The mode switch is connected to pins 6 through 9 allowing the program that is to be run to be chosen. Attached to pins 10 and 11 are the outputs to control the solenoids. Pin 12 is the input capture pin on the microcontroller and is used to take the pulsing input from the Signal IQ from the Radical pulse oximeter. The analog input pin 13 is sued to take the pressure input from the 26PCBFA26 pressure sensor signal conditioning circuit. Pin 14 outputs a signal to control the audible alarm that sounds if an error occurs. The analog Pulse Rate input is connected through signal conditioning circuitry to Pin 15.

The circuits were built on three separate printed circuit boards. The first board contained the microcontroller and the pulse oximeter output signal conditioning circuits. This was because the specified circuits had a 5V supply, and there was a printed 5V line down the center of the microcontroller development board. The second printed circuit board contained the pressure sensor and audible alarm circuits. These circuits all ran on the higher voltage output from the microcontroller of 8.9V. The third board contained the solenoid control circuits including the 5VDC to 120VAC OAC5 relay. The three boards were connected together and placed inside of the case at specified positions.

G. Case Design

With the completion of the vacuum system design and assembly, and the completion of the signal conditioning circuitry design and assembly, a case needed to be designed and constructed to hold all the components of the complete system. The case would have to serve multiple functions. It had to hold all system components, had to be the interface between the user and the device, had to reduce the noise of the system components, had to aid in the cooling of the system circuitry, and it had to present the device in a neat and sanitary fashion. The appearance of the case was critical since the system would be tested and implemented in a hospital.

Based on the components in which the case would be holding the dimensions of 15″×11″×6″ were determined. The case was constructed of ¼″ acrylic, due to ease of manufacturability, strength, and appearance. In order to hide the working components from the user of the system the acrylic was painted white. The white paint was chosen in order to provide a sanitary appearance of the case and it was applied on the inner walls of the case for durability of the finish. The case is supported by four ½″ aluminum feet machined out of 1″ aluminum stock. The feet were drilled and tap to mate with a ¼-20 bolts. The plastic pads were adhered to the feet in to order restrict noise and vibrations from being distributed out of the case.

The case needed to hold the vacuum system components which included the venturi pump and silencer, compressed air solenoid, return to atmosphere solenoid, and provide a spot for the vacuum system to interface with the chamber. It was also necessary for the case to muffle the noise created by the solenoids and vacuum pump. This was achieved by using foam insulators between the mounting brackets of the solenoids and the acrylic which the case was constructed from. The case also needed to provide a spot where the exhaust of the vacuum pump could vent. The vents for the exhaust were position in a way so the exhaust of the vacuum pump would be forced to run over the circuitry, cooling the circuits in the process before the air exits the chamber.

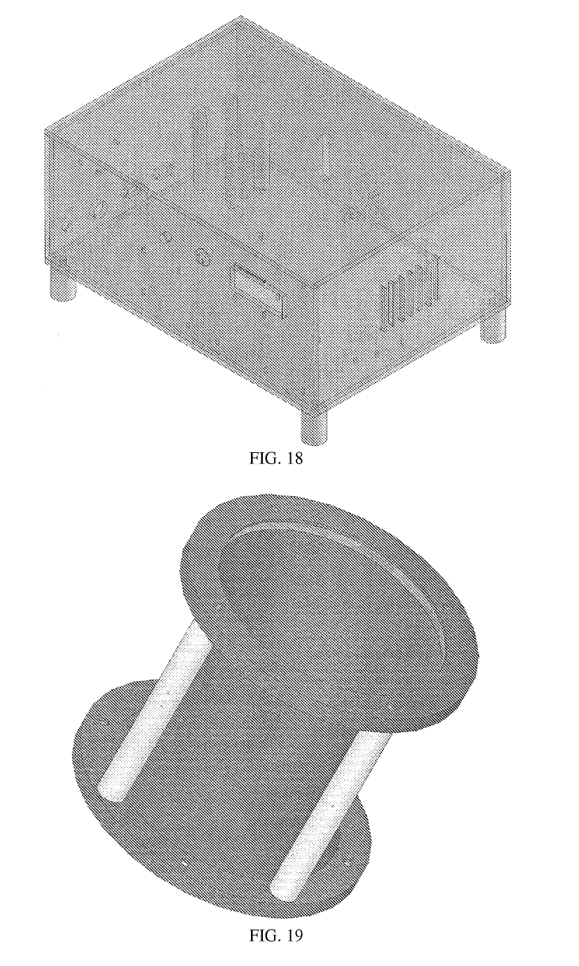

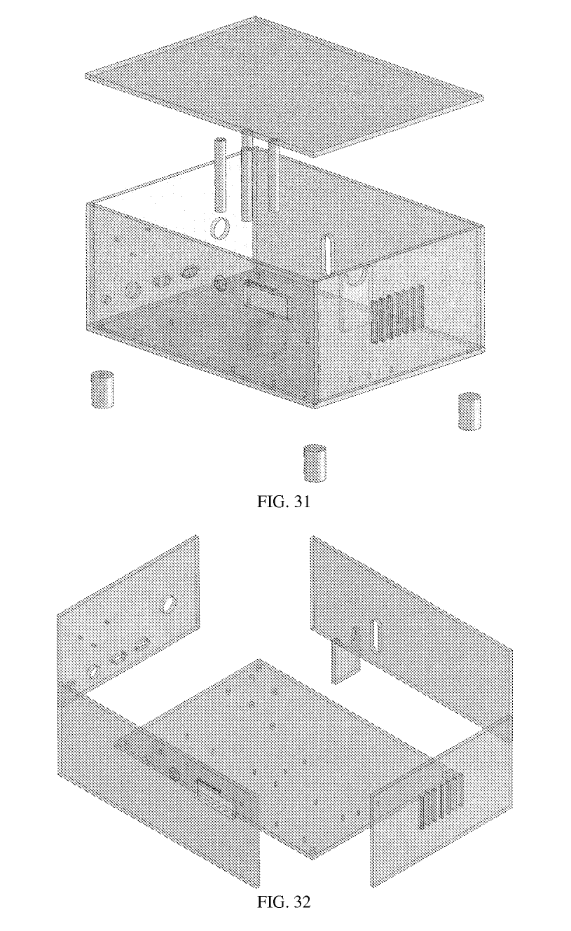

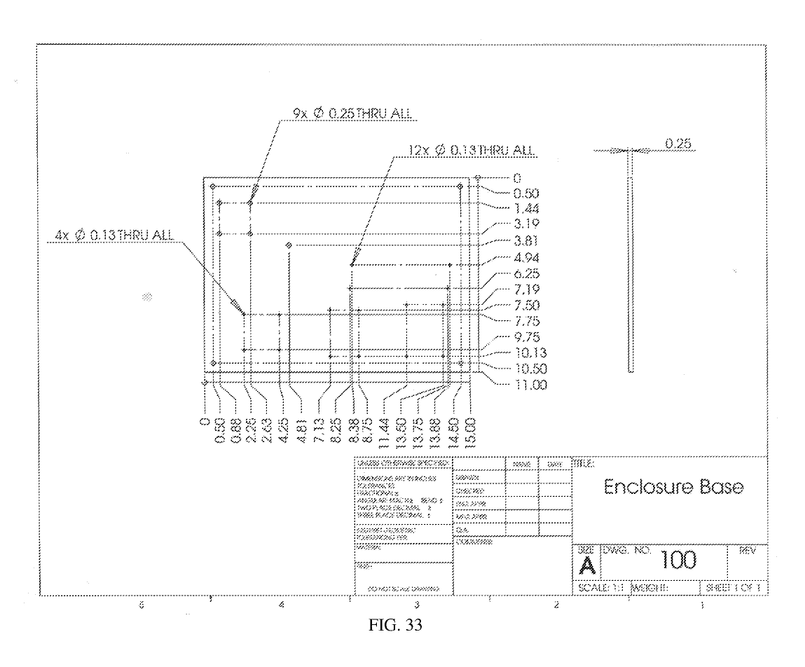

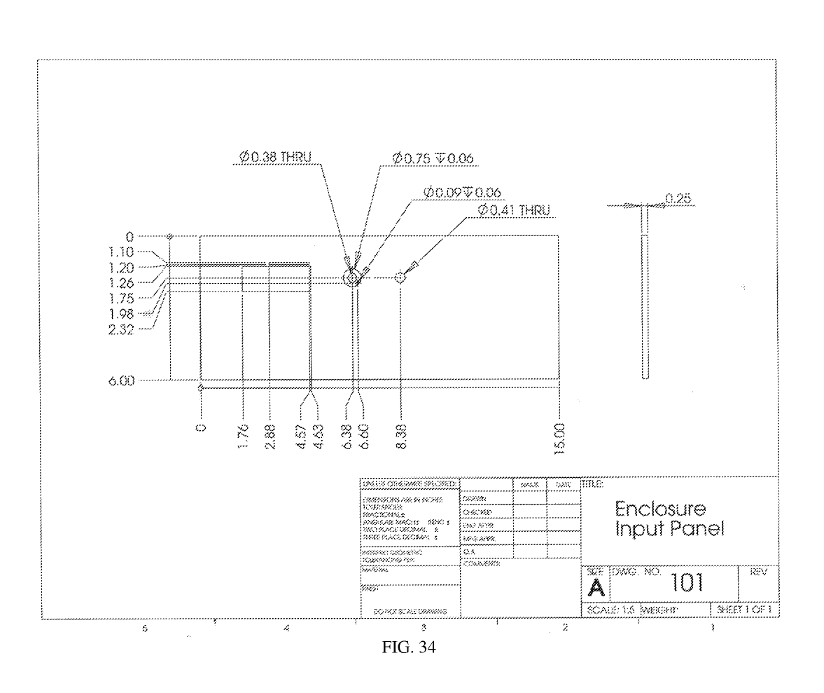

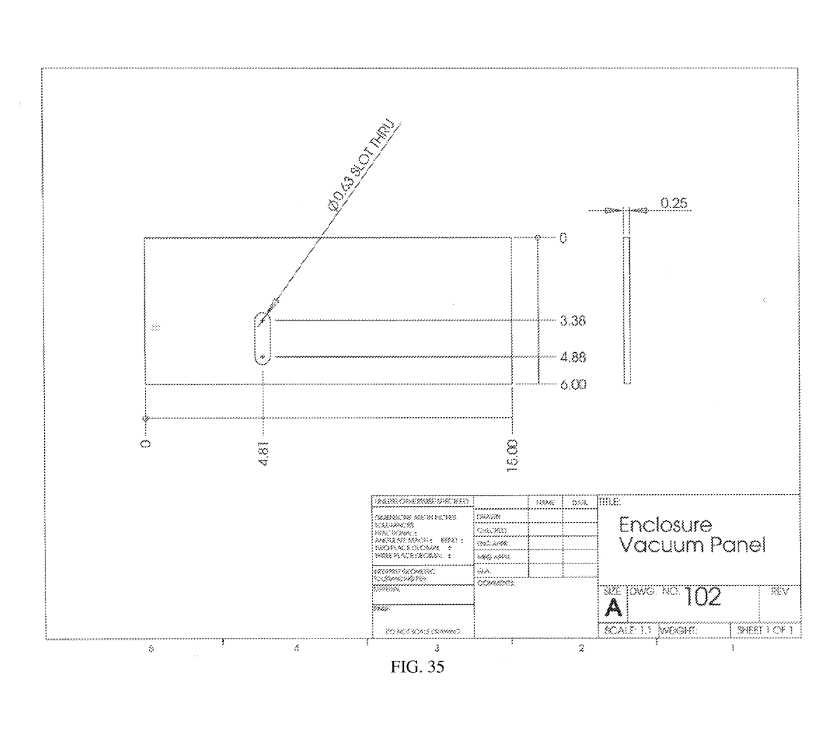

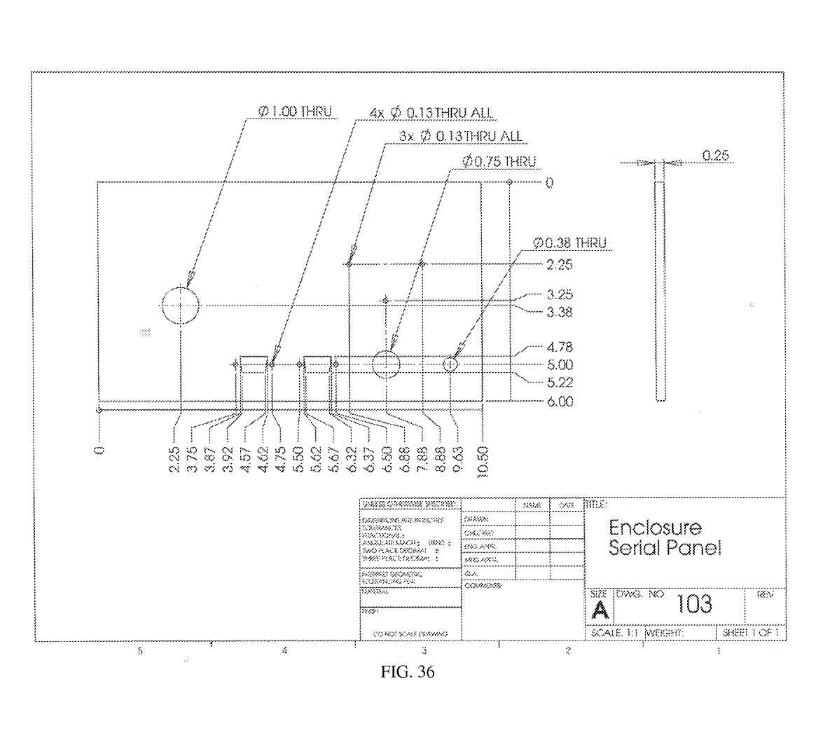

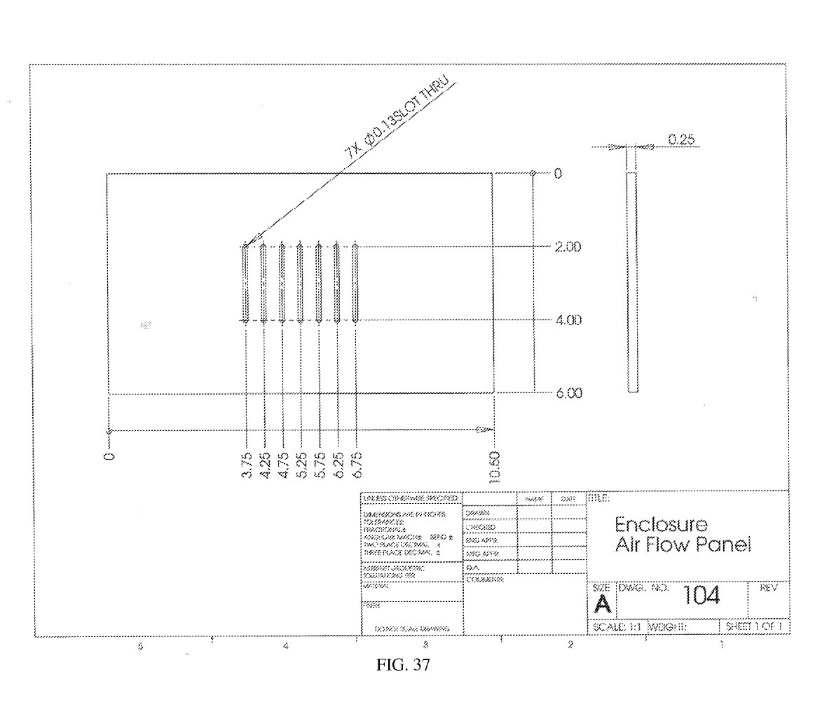

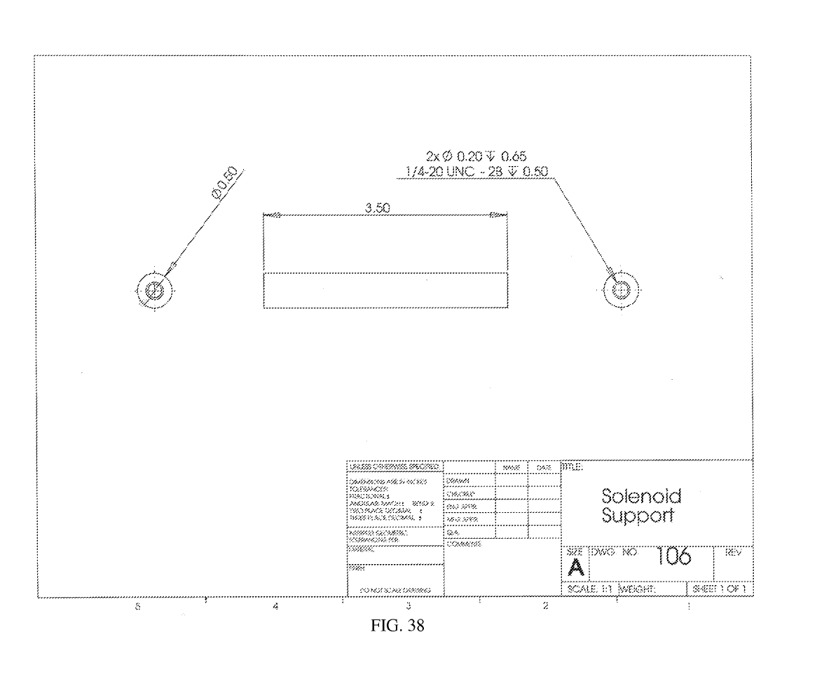

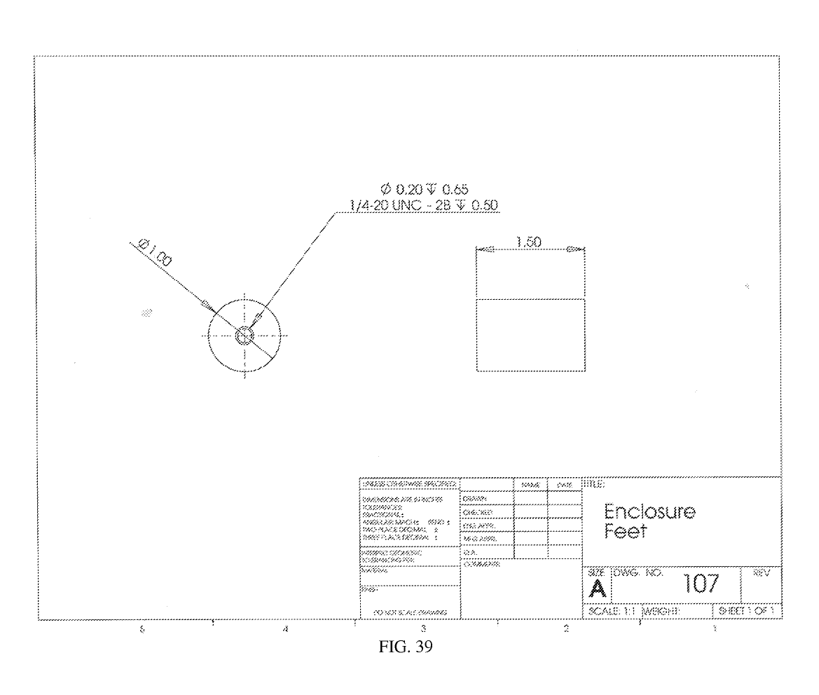

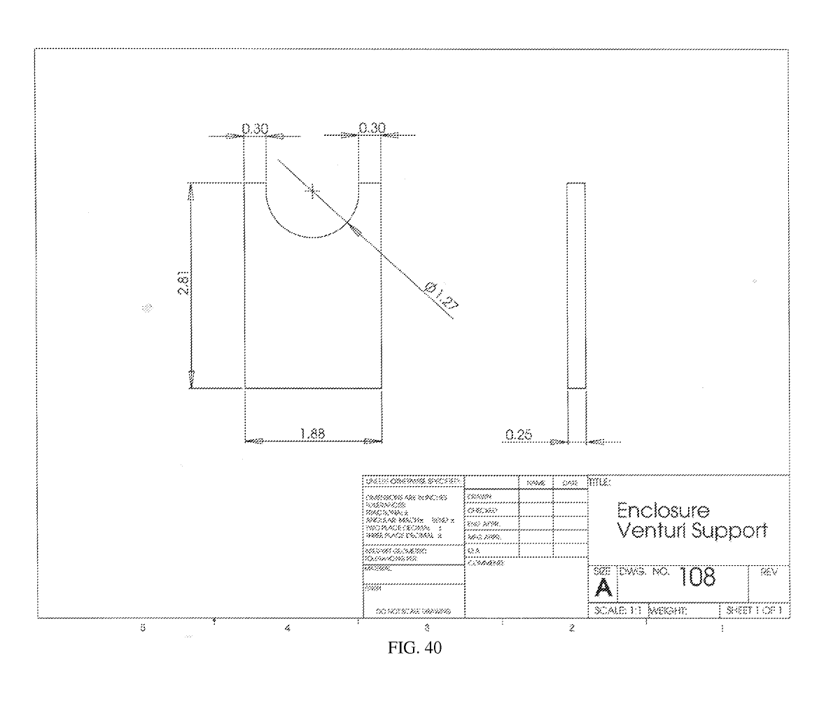

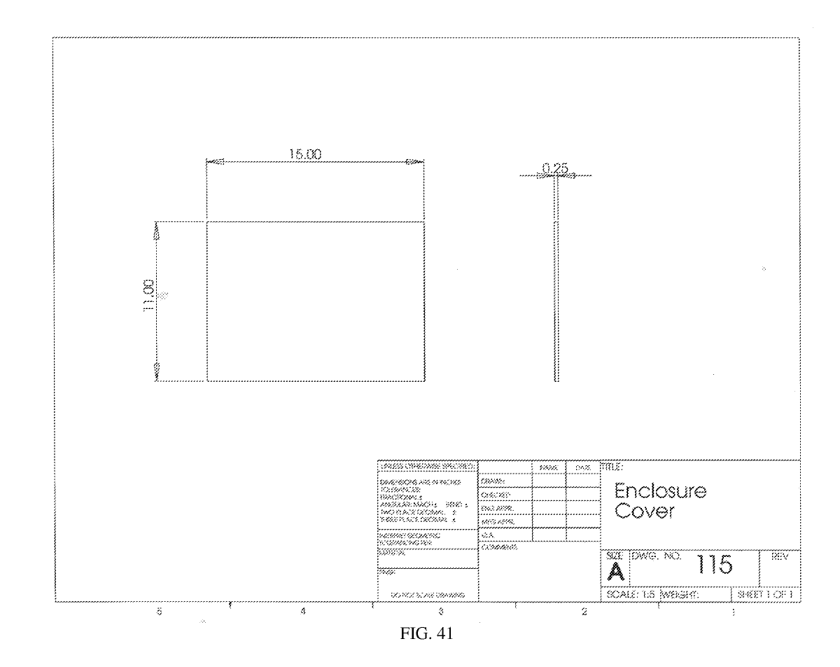

The case also provided the necessary room to mount all electronic components of the system including the microcontroller, signal conditioning circuits, power supply, alarm, serial ports, mode switch, reset switch, pressure sensor, and the on/off switch. The power supply, microcontroller and signal conditioning circuits were enclosed in the case, the rest of the components were placed on the outside of the case to interface the electronic system with the user. For efficiency of use, the LCD screen along with the mode switch, and reset switch were mounted on the front of the case. The serial ports for the pulse oximeter and microcontroller along with the on/off switch, alarm, and power supply were positioned on the same side of the case as the compressed air supply. This side of the case will ideally face the wall of the hospital room. The pressure sensor exits the rear of the case next to the vacuum supply and return lines for the chamber, which will face the patient being treated by the system. The complete case design can be seen in FIG. 18, with more detailed drawings found in FIGS. 31-41.

H. Data Acquisition

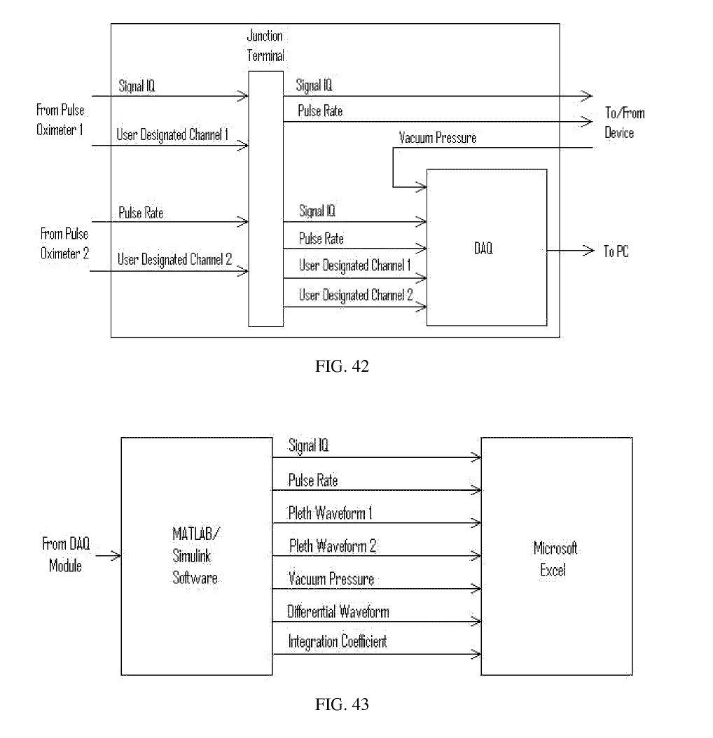

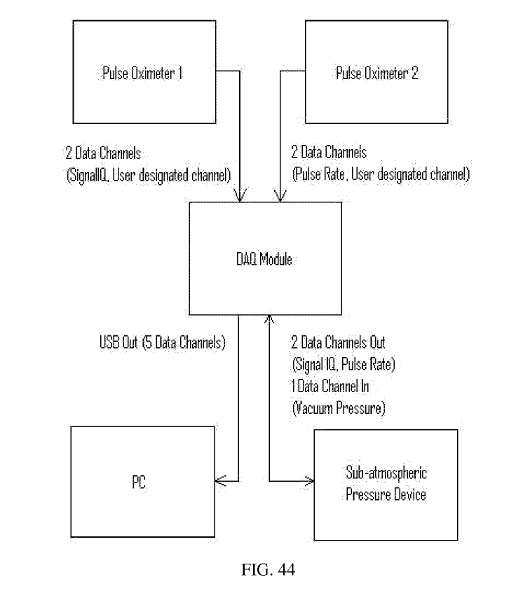

FIGS. 42-44 depict embodiments of data acquisition systems. When the device is used for data acquisition, two pulse oximeters may be used. Each pulse oximeter outputs two channels. Two of the four output channels may be set to output SignalIQ and pulse rate for control purposes. The other two channels can be set however the device operator chooses. For our purposes these channels were set to output the pleth waveform for each hand. All output channels may be routed to a terminal block. The SignalIQ and the pulse rate signals are then relayed to the device for normal control purposes. All channels (5) including a vacuum pressure transducer (used for data only) are routed to a data acquisition module (DAQ) made by Measurement Computing Inc. Through a USB interface, the DAQ is connected to a PC. Using MATLAB software the data is recorded and plotted for a duration specified by the operator. In addition, the difference between pleth waveforms are calculated and integrated. Through a simple programming command all data is exported and saved to Microsoft Excel for further analysis or for future use of oxygenation data. The ability to acquire data on oxygen saturation and the pleth waveform permits fine monitoring and, if desired, adjustment of the treatment device.

Table 8: System Components

TABLE 8
System Components

| | | |
|---|---|---|
| Quan- | | |
| tity | Description | Specifications |

Aluminum Stock

| | | |
|---|---|---|
| 4 | Aluminum feet for case | 1″ × ½″ ¼-20 |
| 4 | Aluminum flange spacers | ½″ × 4″ 4 mm |
| 4 | Aluminum spacers for solenoid | ½″ × 3½″ ¼-20 |

Hardware

| | | |
|---|---|---|
| 12 | Flange hardware, bolts | 4 mm |
| 16 | Flange hardware, washers | 4 mm |
| 4 | Allen head bolts for case feet | ½″ ¼-20 |
| 4 | Hex head bolts for solenoid | ¾″ ¼-20 |
| 8 | Flat washers for solenoid | ¼″ |
| 1 | Bolt for atmosphere solenoid | 6 mm ½″ |
| 1 | Nut for atmosphere solenoid | 6 mm |
| 1 | Washer for atmosphere solenoid | 6 mm |
| 5 | Rubber washers for solenoids | 5 mm |
| 8 | Rubber feet for case | |
| 30 | Stand offs, circuit mounts | |
| 28 | stand off nuts | |
| 6 | Round head bolts case | 6-32 |
| 6 | case nuts | 6-32 |

Vacuum Plumbing

| | | |
|---|---|---|
| 1 | Male compressor fittings | ¼″ NPT |
| 1 | ⅜″ → ¼″ adapter | ⅜″ → ¼″ NPT |
| 1 | ⅜″ thread to thread | ⅜″ NPT |
| 2 | ⅜″ male hose nipples | ⅜″ NPT |
| 3 | ¼″ male hose nipples | ¼″ NPT |
| 1 | ⅜″ NPT nut | ⅜″ NPT |
| 1 | ¼″ NPT nut | ¼″ NPT |
| 2 | ⅜″ washers | ⅜″ |
| 2 | ¼″ washers | ¼″ |
| 6 ft | ⅜″ clear hose | ⅜″ |
| 1 | Teflon tape | |

Acrylic

| | | |
|---|---|---|
| 1 | Acrylic tubing | 3/16″ × 5.5″ × 18″ |
| 1 | Acrylic flat stock | 3/16″ × 11″ × 22″ |
| 1 | Acrylic flat stock | 3/16″ × 6″ × 6″ |
| 2 | Acrylic flat stock | 3/16″ × 18″ × 24″ |
| 1 | Weldon #4 | Methylene Chloride |

Flange Material

| | | |
|---|---|---|
| 1 | Pair of Playtex living gloves | Large |
| 1 | Pair of Playtex living gloves | Medium |
| 1 | Pair of neoprene gloves | Size 7, 30 mills, 16″ |
| 1 | Pair of neoprene gloves | Size 8, 30 mills, 16″ |
| 1 | Pair of neoprene gloves | Size 9, 30 mills, 16″ |

Vacuum Components

| | | |
|---|---|---|
| 1 | Vaccon JF300Venturi pump w/silencer | 12 cfm |
| 1 | Asco ⅜″ compressed air solenoid | 120 V AC |
| 1 | Asco ¼″ vacuum solenoid | 120 V AC |

Electrical Components

| | |
|---|---|
| 2 | OPA340 operational amplifiers |
| 1 | AD620 instrumentation amplifier |
| 3 | 2N3904 BJT transistors |
| 1 | MAX1044 |
| 2 | 10 uF capacitors |
| 2 | OAC5 solid state relays |
| 2 | 10 Watt 2.2 kΩ resistors |
| 8 | 10 kΩ resistors |
| 1 | 50Ω resistor |
| 1 | 5 kΩ potentiometer |
| 1 | 220Ω resistor |
| 1 | 500Ω potentiometer |
| 1 | 26PCBFA26 Honeywell pressure sensor |
| 1 | 6 position switch |
| 4 | 2 MΩ resistor |
| 1 | Audible Alarm(3-12 VDC) |
| 1 | BasicX-24p microcontroller |
| 1 | BasicX-24p development board |
| 2 | Printed circuit boards |
| 1 | Masimo Radical pulse oximeter |
| 1 | 7.5 VDC, 300 mA power supply |

### 4. Discussion

During testing of the pressure sensor a problem was seen from the differential output of the sensor using an 8.9V supply compared to the specification sheet typical value of 10V for the 26PCBFA26 sensor. As can be seen from a comparison of Tables 4 and 5, using a supply voltage of 8.9V caused a loss in the accuracy of the sensor. Previously, it was thought that the pressure sensor would be able to be calibrated through the value of the Rg resistor attached to the AD620 in the signal conditioning circuit seen in FIG. 5. After testing of the sensor the difference from the 10V supply to the 8.9V supply was much greater than expected. The range needed for the sensor was only between 0 psi and 2 psi for the vacuum drop in the chamber. A majority of the devices were not able to go up to the 2 psi range that was desired. Those that were went to much higher pressures, and were not precise in the desired range. Using the IC-11 air regulator only two data points, 0 psi and 1.25 psi, were able to be taken in the optimal range of the system. It was decided that this calibration should wait until a more precise method was developed.

A test was set up to make sure that none of the pieces of the device would break down during long periods of operation. The set up included the completed circuits with the microcontroller program set to control the solenoids using a time based mode. Both solenoids were attached to the circuit. The vacuum solenoid would be open for 5 seconds, and closed for 15 seconds. 55 psi was attached to the input of the Venturi vacuum. No suitable device was found to seal around the flange, so the end of the device that would be enclosed with the hand of the user was left open to atmosphere. The device was checked periodically during the test run. At the completion of the 10 hour run the system was still seen to be running. The 2 10 Watt 2.2 kΩ resistors were felt to be hot. The resistor in parallel with the solenoid at atmosphere was felt to be much hotter then the vacuum solenoid. This is because the resistor in parallel with the solenoid at atmosphere was on for 15 seconds, and the other was on for only 5 seconds. The 10 Watt resistors were dissipating only 6.5 Watts, so they were capable of handling that much power. Because the power is so high it is expected that the resistors would become hot. The problem is not to cause much of a disturbance because the resistors are enclosed inside of the case and will be soldered at a distance from the rest of the electronics.

An initial concern of the project was the noise level that would be associated with the vacuum pump. The chosen Venturi vacuum with attached silencer proved the noise level to not be as much of a concern from the vacuum pump compared to the regenerative blowers and rotary vanes that were initially sought. Once the Venturi vacuum was placed inside of the case the noise level was seen to drop even more. When the solenoids were placed inside of the case the clicking sound caused by opening and closing was heard to increase. This was due to the vibrations inside of the case. The sound was not loud enough to force hearing protection, but it might be loud enough to cause disturbance if placed in a hospital setting.

When the system was completed and running properly initial testing proved very promising for the purpose of the device. The flange was felt to secure an air-tight seal around the arm, but not to restrict venous return by compressing the veins. With an arm placed in the device, and the Radical pulse oximeter reading the pulse, an increase in the area underneath the Pleth waveform on the pulse oximeter was seen to occur. This is very promising, as this area shows that there is an increase in blood flow caused by the drop in pressure synched with the pulse rate around the extremity. Other observations include the hand becoming red if the pressure is on for too long without a release to atmosphere. The fact that the hand is turning red is not good because it means that blood is gathering in the extremity. It shows that the drop in pressure is causing increased blood flow into the device. Vacuum will not be used for this long of durations during actual use of the device. Pulse synchronization will most likely be used, so that the pressure drops with the blood flowing in. As venous return occurs the pressure will return to atmosphere.

### 5. Conclusion

Functional requirements were set to outline the project's direction. From these, a system was developed that would accomplish all of the objectives. The project was broken up into four main areas to design the entire system. A chamber to hold the arm was developed to have minimal size but hold a large variety of arm sizes. To seal around the arm many different flanges were tested. The first was a dishwasher glove that had one side connected to a ring and the other side open. This proved to not hinder the circulation in the arm, but was found to not create a tight seal around the arm. From this, a new design including two rings separated by four bolts with a neoprene flange between the two rings was developed. This proved to properly seal around the arm as well as to not hinder blood flow.

Much time was spent properly developing a vacuum system that would be able to drop the pressure fast enough to cycle with the pulse. To properly accomplish this, knowledge was needed on types of vacuum pumps and solenoids. Needing only a compressed air source to run, the venturi vacuum pump was found to be very quiet and allowed for an extremely fast vacuum source using a solenoid to control the compressed air flow into the pump. Another solenoid was chosen to allow the system to return to atmosphere faster.

A microcontroller based system was used because of flexibility in changing the program. The Radical pulse oximeter was chosen as the method of tissue perfusion measurement because of available outputs through a serial connection. The Signal IQ proved to be an excellent method for cycling the system with a pulse. The Input Capture pin on the microcontroller was found to be the best option for using the Signal IQ. It was possible to calculate the duration of each pulse using the Pulse Rate output of the Radical pulse oximeter through signal conditioning circuitry and into an analog to digital converter on the microcontroller. Using these two outputs a program was written to cycle the pressure in the chamber with the pulse. Two other programs were also written to be tested. These are an alternating pulse mode and time based mode.

From initial testing the prototype appears to support the initial hypothesis that a decrease in pressure around an extremity in a cyclical pattern will increase the blood flow through the extremity. Increases of the Pleth waveform area are signs that there is an increase in blood flow. Other signs are visible changes in the skin color that occur when the device is left with vacuum pressure for a period of time greater than a few seconds. Although the redness is a sign that blood is not being released from the hand, the fact that blood is gathering shows that the drop in pressure is having an effect on blood flow. The blood not being returned to the hand is caused by the drop in pressure being on for too long. This only occurs on the timed based mode when the vacuum cycle is too long.

The control system is adaptive in nature, responding immediately to any change in the patient's pulse, without tweaking and constant supervision by the attending physician. The software allows for many more control options without modification to current circuitry, and allows for a user interface for which the operator does not need in depth knowledge of the device. The data acquisition capabilities can be considered a separate structural difference as they were developed independently from the control system, and most importantly, no prior art makes such mention. Our use of available hospital equipment has resulted in reduced cost, reduced noise, and reduced size. Finally, the flange design does not require any pneumatic cuff or coupling action.

### 6. Variations

A system may include additional controls to provide a simpler and/or more powerful user interface. For example, the interface can be programmed to allow the user to change the ON and OFF times in the timed based mode as well as the pressure amount reached in the chamber. The display may be programmed to show the current mode, the current pressure in the chamber, the time of the running program, and/or the option to change modes.

A wide variety of pressure sensors may be used to monitor pressure in the chamber.

Clicking sounds from the solenoids may be reduced by inserting some sound dampening material around the solenoids and/or between the solenoids and the case.

### 7. Exemplary User Instructions

The following instructions give way to properly operate the device using the pre-installed programs.

A. Device Operation
1. Turn on the Radical pulse oximeter by pressing the on switch on the pulse oximeter.
2. Go to the menu of the device and scroll down to the “OUTPUT” function and select it.
3. Click on “Analog 1” and scroll through the output options until “Signal IQ” is selected.
4. Click on “Analog 2” and scroll though the output options until “Pulse Rate” is selected.
5. Connect the 15 pin serial cable from the back of the pulse oximeter to the 15 pin serial connection on the case labeled “Pulse Oximeter Output.”
6. Plug the device into a 120VAC outlet.
7. Connect a 55 psi compressed air source to the compressed air input on the device.
8. Make sure that the knob on the front of the chamber is set to Standby Mode, Position 1.
9. Turn the device on by setting the red switch to the “ON” position.
10. Attach the Radical pulse oximeter finger clip to the fore finger of the hand that will be placed in the chamber. Allow time for the Radical pulse oximeter to begin reading and displaying the pulse.
11. On the front of the case, turn the knob to the desired position and hit the red reset button. Position 1=Standby Mode (Waits for knob to be turned to a user program). Position 2=Pulse Synchronization Mode (Divides the pulse into pressure reduction and atmospheric sections in the chamber). Position 3=Alternating Pulse Mode (Reduces the pressure for a set number of pulses and then returns to atmosphere for a set number of pulses). Position 4=Time Based Mode (Reduces the pressure for a set amount of time, and then returns to atmosphere for a set amount of time).
12. The system will now cyclically run until the mode switch is turned back to Position 1, and the red reset button is pressed.
13. Once pressure is returned to atmosphere the hand of the user can be easily removed from the chamber.
14. If desired, the finger clip from the Radical pulse oximeter can be removed, and the pulse oximeter turned off.
15. Turn the power off to the device by clicking the red switch to the “OFF” position.

B. Flange Replacement
1. Unbolt the four 4 mm bolts that hold the outer flange ring to the aluminum spacers at the top of the flange, using the appropriate allen-key.
2. Unscrew the aluminum flange spacers; using a 7 mm wrench to hold the 4 mm bolt heads facing the inside of the chamber.
3. Unbolt the remaining four 4 mm bolts that hold the inner flange ring to the chamber using a 7 mm wrench.
4. Once the inner flange ring is removed from the chamber the glove can be unwrapped from the inner and outer flange ring. Note the placement and orientation of the indexes on the flange rings.
5. Cut the hand off of the replacement neoprene glove just below the thumb. Note: The length of the new neoprene flange may be shorter then the used neoprene flange do to plastic deformation, which occurs during use.
6. Once the new neoprene flange is cut to the appropriate length it can be fitted to the inner and outer flange rings.
7. Wrap the end of the neoprene flange which the hand was cut off of, around the inner flange ring.
8. Wrap the opposite side of the neoprene flange around the outer flange ring. Note: inner and outer rings need to be lined up to the marked indexes to align correctly with the chamber.
9. Align the inner flange ring with bolt holes on the chamber.
10. Punch holes through the neoprene flange in the appropriate locations for the 8 4 mm bolts.
11. Replace all eight 4 mm bolts with four of the bolts threads facing out of the chamber and the remaining four facing in the chamber.
12. Replace the nuts and washers on the four 4 mm bolts facing in the chamber using a 7 mm wrench.
13. Replace the aluminum spacers and washers in the operate positions.
14. Punch holes through the neoprene flange in the appropriate locations to hold the outer flange ring to the aluminum spacers.
15. Replace the four 4 mm allen bolts to hold the outer flange ring to the aluminum spacers using the appropriate allen-key.

### BIBLIOGRAPHY

“BasicX-24P Technical Specifications.” Basic X Homepage. 2005. Feb. 18, 2006. <http://www.basicx.com>.
“Radical.” Masimo Corporation Homepage. 2006. Mar. 17, 2006. <http://www.masimo.com/pulseOximeter/radical.htm>.
“Venturi Vacuum Pumps.” Vaccon Vacuum Products. 2004. Jan. 28, 2006. <http://www.vaccon.com/venturi.html>

COMPUTER PROGRAM LISTING

‘Program used to control sub-atmospheric pressure chamber for mechanical assistance of blood flow
‘The program contains 3 modes of operation: Pulse Synchronized, Alternating Pulse, and Time Based
‘Inputs include Masimo Radical Pulse Oximeter, pressure sensor, mode switch, and reset button
‘Outputs include solenoids, audible alarm, and LCD
‘Pins 6,7,8,9 are used for the mode switch
‘Pin 10 is used for the compressed air solenoid; when high, vacuum occurs
‘Pin 11 is used for the atmosphere solenoid; when high, chamber is at atmospheric pressure
‘Pin 12 receives the pulse input from the Radical
‘Pin 13 is used for the pressure sensor
‘Pin 14 is used for the audible alarm
‘Pin 15 receives the pulse rate input from the Radical
‘Define variables
Public Mode as Integer

| | |
|---|---|
| Public PulseTrain as New UnsignedInteger | ‘Unused variable, but must be identified |
| Public Sub Main( ) | |

| | |
|---|---|
| Call StartLCD | ‘Initialize LCD |
| Call PutQueueStr(Com3Out, Chr(Clear_LCD)) | ‘Clear LCD |
| Call PutQueueStr(Com3Out, Chr(Set_Cursor) & Chr(0) & Chr(0)) | ‘Set Cursor on LCD |
| Call PutQueueStr(Com3Out, “Wait for Pulse”) | |
| Do | |

| | |
|---|---|
| Call Delay(0.025) | ‘Delay for Com3 queue to empty |
| Call InputCapture(PulseTrain, 1, 1) | ‘Wait for pulse input |
| Call StartLCD | ‘Re-initialize LCD |
| Call ModeTest | ‘Tests mode switch |
| If (Mode = 0) Then | ‘Calls control module based on mode |

| Call PutQueueStr(Com3Out, Chr(Clear_LCD)) | |
|---|---|
| Call PutQueueStr(Com3Out, Chr(Set_Cursor) & Chr(0) & Chr(0)) | |
| Call PutQueueStr(Com3Out, “Select Mode”) | ‘Asks user to select mode |

| | |
|---|---|
| ElseIf (Mode = 1) Then | ‘Calls Pulse Synchronized Mode |

Call PulseSync

| | |
|---|---|
| ElseIf (Mode = 2) Then | ‘Calls Alternating Pulse Mode |

Call AltPulse

| | |
|---|---|
| ElseIf (Mode = 3) Then | ‘Calls Time Based Mode |

Call TimeOnly

| | |
|---|---|
| Else | ‘Asks for user to select mode |

Call PutQueueStr(Com3Out, Chr(Clear_LCD))
Call PutQueueStr(Com3Out, Chr(Set_Cursor) & Chr(0) & Chr(0))
Call PutQueueStr(Com3Out, “Select Mode”)

End If

Loop

End Sub
‘Initializes LCD screen. This module must be run at the start of the main program,
‘and after the InputCapture command before anything can be displayed.
‘Define Com channels
Public Com3In(1 to 15) as Byte
Public Com3Out(1 to 40) as Byte
‘Define LCD control constraints
Public Const BackLite as Byte = 20
Public Const Clear_LCD as Byte = 12
Public Const Set_Cursor as Byte = 17
Public Sub StartLCD( )

| | |
|---|---|
| Call OpenQueue(Com3In, 15) | ‘Open Com3 Buffers |
| Call OPenQueue(Com3Out, 40) | |
| Call DefineCom3(0, 5, bx1000_1000) | |
| Call OpenCom(3, 9600, Com3In, Com3Out) | ‘Open Com3 Port |
| Call PutQueueStr(Com3Out, Chr(BackLite) & Chr(55)) | ‘Set backlight |

End Sub
‘ModeTest module tests the input from pins 6,7,8,9. Based on switch condition
‘integer is assigned and mode name is displayed.
Public Sub ModeTest( )

| | |
|---|---|
| ‘Define variables | |
| Dim W as Byte | |
| Dim X as Byte | |
| Dim Y as Byte | |
| Dim Z as Byte | |
| ‘Assign pins to variables | |
| W = GetPin(6) | |
| X = GetPin(7) | |
| Y = GetPin(8) | |
| Z = GetPin(9) | |
| If (W = 1) Then | ‘Standby Mode |

Mode = 0
Call PutQueueStr(Com3Out, Chr(Clear_LCD))
Call PutQueueStr(Com3Out, Chr(Set_Cursor) & Chr(0) & Chr(0))
Call PutQueueStr(Com3Out, “Select Mode”)

| | |
|---|---|
| ElseIf (X = 1) Then | ‘Pulse Synchronized Mode |

Mode = 1
Call PutQueueStr(Com3Out, Chr(Clear_LCD))
Call PutQueueStr(Com3Out, Chr(Set_Cursor) & Chr(0) & Chr(0))
Call PutQueueStr(Com3Out, “Pulse Sync. Mode”)

| | |
|---|---|
| ElseIf (Y = 1) Then | ‘Alternating Pulse Mode |

Mode = 2
Call PutQueueStr(Com3Out, Chr(Clear_LCD))
Call PutQueueStr(Com3Out, Chr(Set_Cursor) & Chr(0) & Chr(0))
Call PutQueueStr(Com3Out, “Alt. Pulse Mode”)

| | |
|---|---|
| ElseIf (Z = 1) Then | ‘Time Based Mode |

Mode = 3
Call PutQueueStr(Com3Out, Chr(Clear_LCD))
Call PutQueueStr(Com3Out, Chr(Set_Cursor) & Chr(0) & Chr(0))
Call PutQueueStr(Com3Out, “Time Based Mode”)

End If

End Sub
‘This module contains the Pulse Synchronized Control program.
‘Input received from two analog channels from Radical Pulse Oximeter (PulseRate and Signal IQ)
‘Cycle begins with vacuum in anticipation of SignalIQ pulse. Program then calculates pulse rate
‘and delays. Vacuum continues for specific delay, and then vacuum pressure is tested. Pressure
‘then returns to atmosphere. After delay, pressure is tested. Program then loops to beginning
‘of cycle prior to next Signal IQ pulse.
Public Sub PulseSync( )

| | |
|---|---|
| ‘Define variables | |
| Dim BPM as Single | ‘Pulse Rate (Beats Per Minute) |
| Dim PulseTime as Single | |
| Dim AtmosphereDelay as Single | |
| Dim VacuumDelay as Single | |
| Do | |

| | |
|---|---|
| Call PutPin(11, bxOutputLow) | ‘Close chamber |
| Call PutPin(10, bxOutputHigh) | ‘Open vacuum |
| Call Delay(0.02) | ‘Com3 queue delay |
| Call InputCapture(PulseTrain, 1, 1) | ‘Wait for Signal IQ pulse |
| Call GetADC(15, BPM) | ‘Analog PulseRate input voltage from Radical |
| BPM = 255.0*BPM | ‘Convert voltage to Beats per Minute |
| PulseTime = 60.0/BPM | ‘Calculate time between pulses |
| VacuumDelay = 0.2*PulseTime | ‘Calculates time for remainder of vacuum cycle |
| AtmosphereDelay = 0.5*PulseTime | ‘Calculates time for atmosphere cycle |
| | ‘VacuumDelay + AtmosphereDelay < PulsetTime |
| Call Delay(VacuumDelay) | ‘Delay for vacuum |
| Call VacuumTest | ‘Test pressure |
| Call PutPin(11, bxOutputHigh) | ‘Open chamber |
| Call PutPin(10, bxOutputLow) | ‘Close vacuum |
| Call Delay(AtmosphereDelay) | ‘Delay for atmosphere |
| Call AtmosphereTest | ‘Test pressure |

Loop

End Sub
‘This module contains the Alternating Pulse Control program. Program timing based on
‘Signal IQ pulse signal from Radical Pulse Oximeter. After Signal IQ is received, vacuum
‘occurs until next Signal IQ pulse. At that pulse, chamber returns to atmosphere for 3 pulses
‘signals, or for whatever value is assigned to the variable “Count.” The appropriate pressure
‘is tested for throughout vacuum and atmosphere cycles.
Public Sub AltPulse( )
Do

| | |
|---|---|
| ‘Define variable | |
| Dim Count as Integer | |
| Count = 3 | ‘Assigned number of cycles to remain at atmosphere |
| Call Delay(0.02) | ‘Com3 queue delay |
| Call InputCapture(PulseTrain, 1, 1) | ‘Wait for Signal IQ pulse |
| Call PutPin(11, bxOutputLow) | ‘Close chamber |
| Call PutPin(10, bxOutputHigh) | ‘Open vacuum |
| Call Delay(0.2) | ‘Allow pressure to stabilize |
| Call VacuumTest | ‘Test pressure |
| Do | ‘Atmosphere loop |

| | |
|---|---|
| Call Delay(0.02) | ‘Com3 queue delay |
| Call InputCapture(PulseTrain, 1, 1) | ‘Wait for Signal IQ pulse |
| Call PutPin(11, bxOutputHigh) | ‘Open chamber |
| Call PutPin(10, bxOutputLow) | ‘Close vacuum |
| Call Delay(0.2) | ‘Allow pressure to stabilize |
| Call AtmosphereTest | ‘Test pressure |
| Count = Count − 1 | ‘Decrement Count |

| | |
|---|---|
| Loop While (Count > 0) | ‘Test Count |

Loop
End Sub
‘This module contains the Time Based Control program. This program is independent of the patient
‘pulse. Vacuum and atmosphere cycle time are preset at 5 and 15 seconds respectively. Throughout
‘both cycles the appropriate pressure is tested for.
Public Sub TimeOnly( )

Do

| ‘Define variables | |
|---|---|
| Dim VacuumTime as Single | |
| Dim AtmosphereTime as Single | |
| Dim TestNumber as Single | |
| Dim TestDelay as Single | |
| Dim TestCount as Integer | |
| ‘Assign variables | |
| VacuumTime = 5.0 | ‘Vacuum cycle time |
| AtmosphereTime = 15.0 | ‘Atmosphere cycle time |
| TestNumber = 5.0 | ‘Number of pressure tests per cycle |
| TestCount = 5 | ‘Set test counter |
| ‘TestCount should be the same as TestNumber, if changed, must also be changed below | |
| Call PutPin(11, bxOutputLow) | ‘Close chamber |
| Call PutPin(10, bxOutputHigh) | ‘Open vacuum |

| | |
|---|---|
| Do While (TestCount > 0) | ‘Vacuum test loop |

| | |
|---|---|
| TestDelay = VacuumTime/TestNumber | ‘Calculate delay between tests |
| Call Delay(TestDelay) | ‘Test delay |
| Call VacuumTest | ‘Test pressure |
| TestCount = TestCount − 1 | ‘Decrement test counter |

| | |
|---|---|
| Loop | |
| TestCount = 5 | ‘Reset test counter |
| Call PutPin(11, bxOutputHigh) | ‘Open chamber |
| Call PutPin(10, bxOutputLow) | ‘Close vacuum |
| Do While (TestCount > 0) | ‘Atmosphere test loop |

| | |
|---|---|
| TestDelay = AtmosphereTime/TestNumber | ‘Calculate delay between tests |
| Call Delay(TestDelay) | ‘Test delay |
| Call AtmosphereTest | ‘Test pressure |
| TestCount = TestCount − 1 | ‘Decrement test counter |

Loop

Loop

End Sub
‘This module is used to test pressure during the atmosphere cycle.
‘If vacuum pressure is greater than 25 mmHg, alarm is sounded and error is displayed.
‘Otherwise, alarm is turned off and pressure is displayed as usual.
Public Sub AtmosphereTest( )

| | |
|---|---|
| Call ReadPressure | ‘Reads chamber pressure |
| If (P1 > 25.0) Then | ‘Tests pressure |

| | |
|---|---|
| Call PutPin(14, bxOutputHigh) | ‘Turn on alarm |
| Call StartLCD | ‘Re-initialize LCD |

Call PutQueueStr(Com3Out, Chr(Set_Cursor) & Chr(1) & Chr(0))
Call PutQueueStr(Com3Out, “Error3: ”& Mid(ASCII_P1, 1, 4) & “mmHg”)

‘Display error and pressure

Else

| | |
|---|---|
| Call PutPin(14, bxOutputLow) | ‘Turn off alarm |
| Call DisplayPressure | ‘Display pressure |

End If

End Sub
‘This module is used to test pressure during the vacuum cycle. If vacuum pressure is
‘outside the range of 25 to 150 mmHg, the alarm is sounded and error is displayed.
‘Otherwise, alarm is turned off and pressure is displayed as usual.
Public Sub VacuumTest( )

| | |
|---|---|
| Call ReadPressure | ‘Reads chamber pressure |
| If (P1 < 25.0) Then | ‘Tests pressure |

| | |
|---|---|
| Call PutPin(14, bxOutputHigh) | ‘Turn on alarm |
| Call StartLCD | ‘Re-initialize LCD |

Call PutQueueStr(Com3Out, Chr(Set_Cursor) & Chr(1) & Chr(0))
Call PutQueueStr(Com3Out, “Error1: ”& Mid(ASCII_P1, 1, 4) & “mmHg”)

| | ‘Display error and pressure |
|---|---|
| ElseIf (P1 > 150.0) Then | ‘Tests pressure |

| | |
|---|---|
| Call PutPin(14, bxOutputHigh) | ‘Turn on alarm |
| Call StartLCD | ‘Re-initialize LCD |

Call PutQueueStr(Com3Out, Chr(Set_Cursor) & Chr(1) & Chr(0))
Call PutQueueStr(Com3Out, “Error2: ”& Mid(ASCII_P1, 1, 4) & “mmHg”)

‘Display error and pressure

Else

| | |
|---|---|
| Call PutPin(14, bxOutputLow) | ‘Turn off alarm |
| Call DisplayPressure | ‘Displays pressure |

End If

End Sub
‘This module is used to read pressure from the sensor through the A/D converter.
‘Input voltage is converted to a value for pressure in mmHg.
‘Also, the pressure value is converted to a string so that it can be displayed on the LCD.
‘Define pressure variable
Public P1 as Single
‘Set Pressure to ASCII to be displayed
Public ASCII_P1 as String * 4
Public Sub ReadPressure( )

| | |
|---|---|
| Call GetADC(13, P1) | ‘Convert analog input voltage |
| P1 = 258.6*P1 | ‘Calculate pressure in mmHg |
| ASCII_P1 = Cstr(P1) | ‘Convert pressure to string |

End Sub
‘This module is used to display pressure in mmHg when no errors occur.
Public Sub DisplayPressure( )

| | |
|---|---|
| StartLCD | ‘Re-initialize LCD |

Call PutQueueStr(Com3Out, Chr(Set_Cursor) & Chr(1) & Chr(0))
Call PutQueueStr(Com3Out, “Vacuum: ”& Mid(ASCII_P1, 1, 4) & “mmHg”)

‘Display vacuum pressure in mmHg

End Sub

1. A subatmospheric pressure treatment device comprising:
a chamber sized to receive an extremity of a subject, the chamber comprising:
a housing having an interior space and being so formed from one or more materials and so sized and shaped as to hold up to a subatmospheric pressure in the interior space of the chamber of at least 25 mmHg below an ambient pressure outside the chamber, the interior space being so sized and shaped as to receive the extremity of the subject;
an inlet communicating with the interior space of the chamber, the inlet so sized and shaped as to pass the extremity of the subject;
a flange attached to the chamber housing and defining the inlet, the flange comprising:
at least two supporting rings affixed to one another by at least one strut; and
a sleeve of material having two ends, each end mounted to a respective one of the supporting rings, the sleeve being:
so pliable as to:
form an air-tight seal around the extremity of the subject when the interior space of the chamber is at the subatmospheric pressure; and
exert essentially no pressure on the extremity of the patient when the interior space of the chamber is at the ambient pressure outside the chamber; and
so rigid as to resist involution into the interior space of the chamber when the interior space of the chamber is at the subatmospheric pressure;
a vacuum source communicating with the chamber, the vacuum source having sufficient capacity to produce the subatmospheric pressure in the interior space of the chamber;
a pulse sensor configured to produce a signal indicative of a pulse waveform representative of the subject's pulse; and
a controller coupled to the vacuum source and to the pulse sensor and so configured as to command the vacuum source, in response to the signal indicative of the pulse waveform, to achieve the subatmospheric pressure in the interior space of the chamber.

2. The device of claim 1, further comprising a pressure gauge in communication with the interior space of the chamber to measure pressure in the interior space.

3. The device of claim 1, further comprising a data acquisition system so coupled to the pulse sensor as to receive one or more signals indicative of at least one of the subject's pulse rate, pulse occurrences, and an oxygenation state of the subject's blood.

4. The device of claim 1, wherein the sleeve material comprises neoprene.

5. The device of claim 1, wherein the vacuum source has sufficient capacity to lower the interior space pressure from the ambient pressure to the subatmospheric pressure in at most 0.1 seconds.

6. The device of claim 1, wherein the controller is so configured as to command the vacuum source to achieve the subatmospheric pressure in the interior space of the chamber in synchrony with a systolic portion of the subject's pulse as sensed by the pulse sensor.

7. The device of claim 6, wherein the controller is so configured as to command the vacuum source to achieve the subatmospheric pressure in the interior space of the chamber with each pulse.

8. The device of claim 6, wherein the controller is so configured as to command the vacuum source to achieve the subatmospheric pressure in the interior space of the chamber with every other pulse.

9. The device of claim 6, wherein the controller is so configured as to command the vacuum source to restore the interior space of the chamber to ambient pressure in between each command to achieve the subatmospheric pressure.

10. The device of claim 9, wherein the controller is so configured as to command the vacuum source to restore the interior space of the chamber to ambient pressure at about the start of a diastolic portion of the subject's pulse as sensed by the pulse sensor.

11. The device of claim 6, wherein the controller is so configured as to command the vacuum source to achieve the subatmospheric pressure in the interior space of the chamber over the course of two or more pulses and then to restore the interior space of the chamber to ambient pressure for two or more pulses.

12. The device of claim 6, wherein the controller is so configured as to command the vacuum source to achieve the subatmospheric pressure in the interior space of the chamber at about the start of the systolic portion of the subject's pulse as sensed by the pulse sensor.

13. The device of claim 1, wherein the subatmospheric pressure is at least 75 mm Hg below the ambient pressure.

14. The device of claim 1, wherein the subatmospheric pressure is at least 100 mm Hg below the ambient pressure.

15. The device of claim 1, wherein the pulse sensor comprises a pulse oximeter.

16. A subatmospheric pressure treatment device comprising:
a chamber sized to receive an extremity of a subject, the chamber comprising:
a housing having an interior space and being so formed from one or more materials and so sized and shaped as to hold up to a subatmospheric pressure in the interior space of the chamber of at least 25 mmHg below an ambient pressure outside the chamber, the interior space being so sized and shaped as to receive the extremity of the subject;
an inlet communicating with the interior space of the chamber, the inlet so sized and shaped as to pass the extremity of the subject and to seal around the subject when the interior space of the chamber is at the subatmospheric pressure;
a vacuum source communicating with the chamber, the vacuum source having sufficient capacity to produce a pressure in the interior space of the chamber that is at least 25 mmHg below the ambient pressure outside the chamber;
a pulse sensor configured to produce a signal indicative of a pulse waveform representative of the subject's pulse; and
a microcontroller coupled to the vacuum source and to the pulse sensor and so programmed as to command the vacuum source, in response to the signal indicative of the pulse waveform, to achieve a pressure in the interior space of the chamber that is at least 25 mmHg below the ambient pressure outside the chamber.

17. A method of increasing blood flow in a subject's extremity using the device of claim 1, the method comprising:
affixing a pulse sensor to the extremity;
passing the subject's extremity through the inlet and into the interior space of the chamber;
sensing the subject's pulse; and
causing the controller to command the vacuum source to alternately achieve the ambient pressure and the subatmospheric pressure in the interior space of the chamber in response to the sensed pulse.

18. The method of claim 17, wherein the subject's pulse is detected when the extremity is in a low-perfusion state.

19. The method of claim 17, wherein the subatmospheric pressure is at least 100 mm Hg below the ambient pressure.

20. (canceled)
